(19) United States
(12) Patent Application Publication
(10) Pub. No.: US 2014/0039317 A1
(43) Pub. Date: Feb. 6, 2014

(54) ULTRASOUND DIAGNOSIS APPARATUS AND CONTROLLING METHOD

(71) Applicants: TOSHIBA MEDICAL SYSTEMS CORPORATION, Otawara-shi (JP); KABUSHIKI KAISHA TOSHIBA, Minato-ku (JP)

(72) Inventor: Takeshi SATO, Tochigi (JP)

(73) Assignees: TOSHIBA MEDICAL SYSTEMS CORPORATION, Otawara-shi (JP); KABUSHIKI KAISHA TOSHIBA, Minato-ku (JP)

(21) Appl. No.: 14/039,972

(22) Filed: Sep. 27, 2013

Related U.S. Application Data
(63) Continuation of application No. PCT/JP2013/070813, filed on Jul. 31, 2013.

(30) Foreign Application Priority Data
Jul. 31, 2012 (JP) 2012-169997
Jul. 31, 2013 (JP) 2013-159663

(51) Int. Cl. A61B 8/00; A61B 8/08

(52) U.S. Cl. CPC A61B 8/54; A61B 8/488; A61B 8/485; A61B 8/5207; A61B 8/5246
USPC 600/443

(57) ABSTRACT

An ultrasound diagnosis apparatus includes: an ultrasound probe that transmits and receives ultrasound waves; and a controlling unit that causes the ultrasound probe to perform a first ultrasound scan to obtain information related to motion of a moving object within a first scanned region and that, as a second ultrasound scan to obtain information about a tissue form within a second scanned region, causes the ultrasound probe to perform an ultrasound scan in each of sectioned regions into which the second scanned region is divided, in a time-division manner between the first ultrasound scans. As the first ultrasound scan, the controlling unit causes the ultrasound scan to be performed according to a method for obtaining the information related to the motion of the moving object by which a high pass filtering process is performed along a frame direction on reception signals obtained from scanning lines structuring the first scanned region.

### CROSS-REFERENCE TO RELATED APPLICATIONS

This application is a continuation of PCT international application Ser. No. PCT/JP2013/070813 filed on Jul. 31, 2013 which designates the United States, incorporated herein by reference, and which claims the benefit of priority from Japanese Patent Application No. 2012-169997 filed on Jul. 31, 2012; and Japanese Patent Application No. 2013-159663, filed on Jul. 31, 2013, the entire contents of which are incorporated herein by reference.

### FIELD

Embodiments described herein relate generally to an ultrasound diagnosis apparatus and a controlling method.

### BACKGROUND

Conventionally, to perform an ultrasound image diagnosis process, a method is known by which images indicating moving object information (e.g., bloodstream images such as color Doppler images) are subject to an imaging process at a high frame rate. Further, conventionally, to perform an ultrasound image diagnosis process, it is common practice to display, for example, tissue images (B-mode images) and bloodstream images at the same time.

When B-mode images and bloodstream images are displayed at the same time by using such a conventional method, however, in order to display the bloodstream images at a high frame rate, with little noise, and with a high sensitivity, it is necessary to generate and display the B-mode images from reception signals used for obtaining bloodstream information, without performing a B-mode-exclusive scan. As a result, image quality of the tissue images is degraded in some situations, for example, because the reception signals saturate, because the density of scanning lines is low, or because it is not possible to perform tissue harmonic imaging.

### BRIEF DESCRIPTION OF THE DRAWINGS

FIG. 1 is a block diagram of an exemplary configuration of an ultrasound diagnosis apparatus according to a first embodiment;

FIG. 2 is a drawing of exemplary processes performed by a B-mode processing unit;

FIG. 3 is a block diagram of an exemplary configuration of the Doppler processing unit shown in FIG. 1;

FIG. 4 is a drawing for explaining a wall filtering process performed by implementing a high frame rate method;

FIG. 5A and FIG. 5B are drawings for explaining examples of conventional methods;

FIG. 6 is a drawing of an example of a problem with the conventional methods;

FIG. 7 and FIG. 8 are drawings for explaining a controlling unit according to the first embodiment;

FIG. 9A and FIG. 9B are drawings of examples of display modes according to the first embodiment;

FIG. 10 is a flowchart for explaining an example of an ultrasound scan controlling process performed by the ultrasound diagnosis apparatus according to the first embodiment;

FIG. 11 is a drawing for explaining a second embodiment;

FIG. 12 is a flowchart for explaining an example of an output controlling process performed by an ultrasound diagnosis apparatus according to the second embodiment;

FIG. 13A and FIG. 13B are drawings for explaining a third embodiment;

FIG. 14A and FIG. 14B are drawings for explaining a fourth embodiment; and

FIG. 15, FIG. 16 and FIG. 17 are drawings for explaining a fifth embodiment.

### DETAILED DESCRIPTION

An ultrasound diagnostic apparatus according to an embodiment includes an ultrasound probe and a controlling unit. The ultrasound probe is configured to transmit and receive an ultrasound wave. The controlling unit is configured to cause the ultrasound probe to perform a first ultrasound scan to obtain information related to motion of a moving object within a first scanned region and causes the ultrasound probe to perform, as a second ultrasound scan to obtain information about a tissue form within a second scanned region, an ultrasound scan in each of a plurality of sectioned regions into which the second scanned region is divided, in a time-division manner between the first ultrasound scans. As the first ultrasound scan, the controlling unit causes the ultrasound scan to be performed according to a method for obtaining the information related to the motion of the moving object by which a high pass filtering process is performed along a frame direction on reception signals obtained from a plurality of scanning lines structuring the first scanned region.

Exemplary embodiments of an ultrasound diagnosis apparatus will be explained in detail below, with reference to the accompanying drawings.

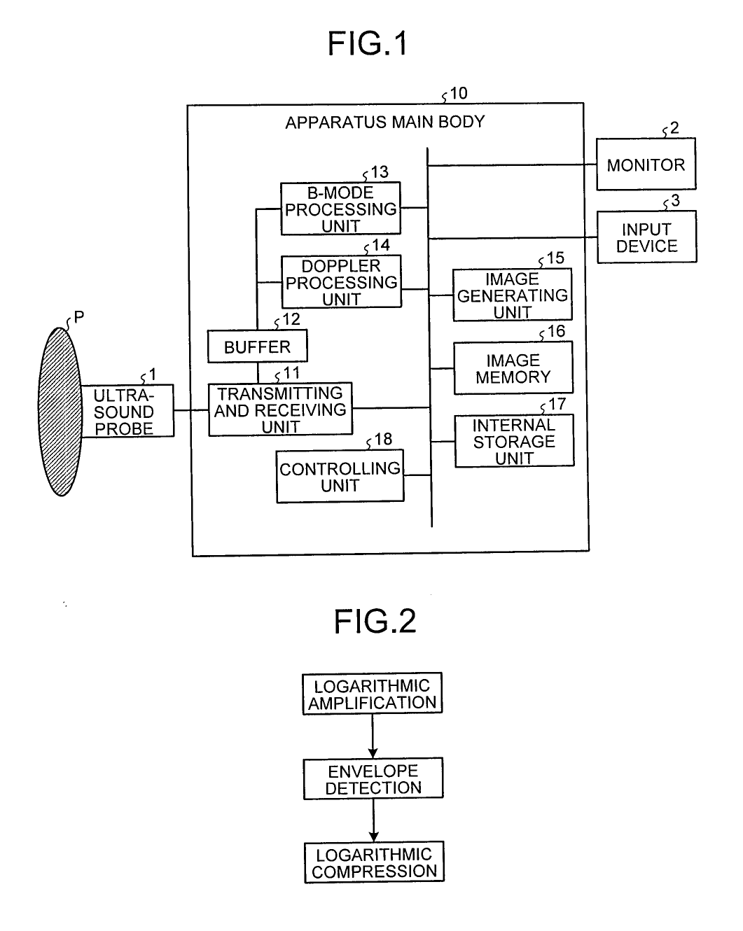

First, a configuration of an ultrasound diagnosis apparatus according to a first embodiment will be explained. FIG. 1 is a block diagram of an exemplary configuration of the ultrasound diagnosis apparatus according to the first embodiment. As shown in FIG. 1, the ultrasound diagnosis apparatus according to the first embodiment includes an ultrasound probe 1, a monitor 2, an input device 3, and an apparatus main body 10.

The ultrasound probe 1 is connected to the apparatus main body 10 to transmit and receive ultrasound waves. For example, the ultrasound probe 1 includes a plurality of piezoelectric transducer elements, which generate an ultrasound wave based on a drive signal supplied from a transmitting and receiving unit 11 included in the apparatus main body 10 (explained later). Further, the plurality of piezoelectric transducer elements included in the ultrasound probe 1 receive reflected waves from a subject P and convert the received reflected waves into electric signals. Further, the ultrasound probe 1 includes matching layers included in the piezoelectric transducer elements, as well as a backing member that prevents ultrasound waves from propagating rearward from the piezoelectric transducer elements. The ultrasound probe 1 is detachably connected to the apparatus main body 10.

When an ultrasound wave is transmitted from the ultrasound probe 1 to the subject P, the transmitted ultrasound wave is repeatedly reflected on a surface of discontinuity of acoustic impedances at a tissue in the body of the subject P and is received as a reflected-wave signal by the plurality of piezoelectric transducer elements included in the ultrasound probe 1. The amplitude of the received reflected-wave signal is dependent on the difference between the acoustic impedances on the surface of discontinuity on which the ultrasound wave is reflected. When a transmitted ultrasound pulse is reflected on the surface of a flowing bloodstream or a moving cardiac wall, the reflected-wave signal is, due to the Doppler effect, subject to a frequency shift, depending on a velocity component of the moving objects with respect to the ultrasound wave transmission direction.

The first embodiment is applicable to a situation where the ultrasound probe 1 is a one-dimensional (1D) array probe configured to scan the subject P two-dimensionally and to a situation where the ultrasound probe 1 is a mechanical four-dimensional (4D) probe or a two-dimensional (2D) array probe configured to scan the subject P three-dimensionally.

The input device 3 includes a mouse, a keyboard, a button, a panel switch, a touch command screen, a foot switch, a trackball, a joystick, and the like. The input device 3 receives various types of setting requests from an operator of the ultrasound diagnosis apparatus and transfers the received various types of setting requests to the apparatus main body 10.

The monitor 2 displays a Graphical User Interface (GUI) used by the operator of the ultrasound diagnosis apparatus to input the various types of setting requests through the input device 3 and displays ultrasound image data and the like generated by the apparatus main body 10.

The apparatus main body 10 is an apparatus configured to generate ultrasound image data based on the reflected-wave signal received by the ultrasound probe 1. The apparatus main body 10 shown in FIG. 1 is an apparatus configured to be able to generate two-dimensional ultrasound image data based on a two-dimensional reflected-wave signal and to be able to generate three-dimensional ultrasound image data based on a three-dimensional reflected-wave signal. It should be noted, however, that the first embodiment is also applicable to a situation where the apparatus main body 10 is an apparatus exclusively for two-dimensional data.

As shown in FIG. 1, the apparatus main body 10 includes the transmitting and receiving unit 11, a buffer 12, a B-mode processing unit 13, a Doppler processing unit 14, an image generating unit 15, an image memory 16, an internal storage unit 17, and a controlling unit 18.

The transmitting and receiving unit 11 is configured to control ultrasound transmissions and receptions performed by the ultrasound probe 1, on the basis of an instruction from the controlling unit 18 (explained later). The transmitting and receiving unit 11 includes a pulse generator, a transmission delaying circuit, a pulser, and the like and supplies the drive signal to the ultrasound probe 1. The pulse generator repeatedly generates a rate pulse for forming a transmission ultrasound wave at a predetermined repetition frequency called a Pulse Repetition Frequency (PRF). Further, the transmission delaying circuit applies a delay period that is required to converge the ultrasound wave generated by the ultrasound probe 1 into the form of a beam and to determine transmission directionality and that corresponds to each of the piezoelectric transducer elements, to each of the rate pulses generated by the pulse generator. Further, the pulser applies a drive signal (a drive pulse) to the ultrasound probe 1 with timing based on the rate pulses. In other words, the transmission delaying circuit arbitrarily adjusts the transmission directions of the ultrasound waves transmitted from the piezoelectric transducer element surfaces, by varying the delay periods applied to the rate pulses.

The transmitting and receiving unit 11 has a function to be able to instantly change the transmission frequency, the transmission drive voltage, and the like, for the purpose of executing a predetermined scanning sequence based on an instruction from the controlling unit 18 (explained later). In particular, the configuration to change the transmission drive voltage is realized by using a linear-amplifier-type transmitting circuit of which the value can be instantly switched or by using a mechanism configured to electrically switch between a plurality of power source units.

Further, the transmitting and receiving unit 11 includes an amplifying circuit, an Analog/Digital (A/D) converter, a reception delaying circuit, an adder, a quadrature detection circuit, and the like and generates reflected-wave data by performing various types of processes on the reflected-wave signal received by the ultrasound probe 1. The amplifying circuit amplifies the reflected-wave signal for each of channels and performs a gain correcting process. The A/D converter applies an A/D conversion to the gain-corrected reflected-wave signal. The reception delaying circuit applies a reception delay period required to determine reception directionality to the digital data. The adder performs an adding process on the reflected-wave signals to which the reception delay periods have been applied by the reception delaying circuit. As a result of the adding process performed by the adder, reflected components from the direction corresponding to the reception directionality of the reflected-wave signals are emphasized.

Further, the quadrature detection circuit converts the output signal from the adder into an in-phase signal (an “I signal”) and a quadrature-phase signal (a “Q signal”) in a baseband bandwidth. After that, the quadrature detection circuit stores the I signal and the Q signal (hereinafter, “IQ signals”) into the buffer 12 as the reflected-wave data. Alternatively, the quadrature detection circuit may convert the output signal from the adder into Radio Frequency (RF) signals and store the RF signals into the buffer 12. The IQ signals or the RF signals serve as signals (reception signals) containing phase information. In the following sections, the reflected-wave data output by the transmitting and receiving unit 11 may be referred to as “reception signals”.

When a two-dimensional scan is performed on the subject P, the transmitting and receiving unit 11 causes the ultrasound probe 1 to transmit two-dimensional ultrasound beams. The transmitting and receiving unit 11 then generates two-dimensional reflected-wave data from the two-dimensional reflected-wave signals received by the ultrasound probe 1. In contrast, when a three-dimensional scan is performed on the subject P, the transmitting and receiving unit 11 causes the ultrasound probe 1 to transmit three-dimensional ultrasound beams. The transmitting and receiving unit 11 then generates three-dimensional reflected-wave data from the three-dimensional reflected-wave signals received by the ultrasound probe 1.

Further, the transmitting and receiving unit 11 is able to generate reflected-wave data having a plurality of reception focuses, from the reflected-wave signals at the piezoelectric transducer elements obtained from one-time ultrasound beam transmission. In other words, the transmitting and receiving unit 11 is a circuit capable of performing a parallel simultaneous reception process. The first embodiment, however, is also applicable to a situation where the transmitting and receiving unit 11 is not capable of performing a parallel simultaneous reception process.

The buffer 12 is a buffer configured to temporarily store therein the reflected-wave data (the IQ signals) generated by the transmitting and receiving unit 11. More specifically, the buffer 12 stores therein the IQ signals corresponding to a certain number of frames or the IQ signals corresponding to a certain number of volumes. For example, the buffer 12 may be a First-In/First-Out (FIFO) memory configured to store therein the IQ signals corresponding to a predetermined number of frames. Further, for example, when the transmitting and receiving unit 11 has newly generated IQ signals corresponding to one frame, the buffer 12 discards the IQ signals corresponding to one frame that were generated earliest and stores therein the newly-generated IQ signals corresponding to the one frame.

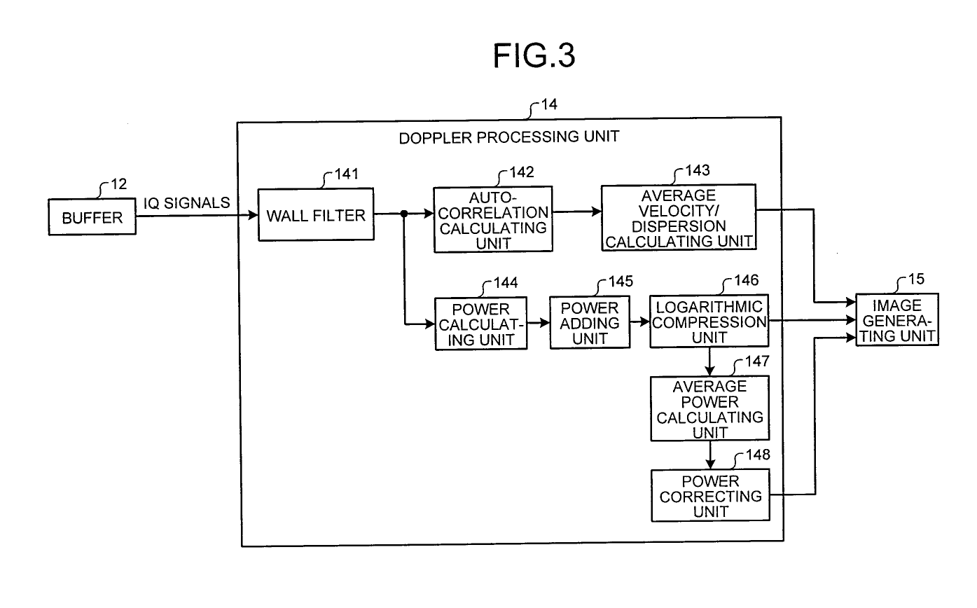

The B-mode processing unit 13 and the Doppler processing unit 14 are signal processing units configured to perform various types of signal processing processes on the reflected-wave data generated from the reflected-wave signals by the transmitting and receiving unit 11. FIG. 2 is a drawing of exemplary processes performed by the B-mode processing unit. As shown in FIG. 2, the B-mode processing unit 13 generates data (B-mode data) in which the strength of each of the signals at multiple points is expressed by a degree of brightness, by performing a logarithmic amplification, an envelope detection process, a logarithmic compression, and the like on the reflected-wave data (the IQ signals) read from the buffer 12.

By performing a filtering process, the B-mode processing unit 13 is able to change the frequency band subject to the imaging process, by varying the detected frequency. By using the filtering process function of the B-mode processing unit 13, it is possible to perform a harmonic imaging process such as a contrast harmonic imaging (CHI) process or a Tissue Harmonic Imaging (THI) process. In other words, from the reflected-wave data of the subject P into whom a contrast agent has been injected, the B-mode processing unit 13 is able to separate reflected-wave data (harmonic data or subharmonic data) of a harmonic component of which the contrast agent (microbubbles and bubbles) serves as sources of the reflections and reflected-wave data (fundamental wave data) of a fundamental wave component of which a tissue inside the subject P serves as sources of the reflections. From the reflected-wave data (the reception signals) of the harmonic component, the B-mode processing unit 13 is able to generate B-mode data from which contrast-enhanced image data is to be generated.

Further, by using the filtering process function of the B-mode processing unit 13, it is possible to separate the harmonic data or the subharmonic data, which is the reflected-wave data (the reception signals) of the harmonic component, from the reflected-wave data of the subject P, by performing the Tissue Harmonic Imaging (THI) process. Then, from the reflected-wave data (the reception signals) of the harmonic component, the B-mode processing unit 13 is able to generate B-mode data from which tissue image data excluding noise components is to be generated.

Furthermore, when performing a harmonic imaging process such as a CHI process or a THI process, the B-mode processing unit 13 is also able to extract the harmonic component by using a method different from the method described above employing the filtering process. During a harmonic imaging process, any of the following imaging methods may be implemented: an Amplitude Modulation (AM) method, a Phase Modulation (PM) method, and an AMPM method combining the AM method and the PM method. According to the AM method, the PM method, and the AMPM method, an ultrasound transmission is performed multiple times on any one scanning line while varying the amplitude and/or the phase thereof. As a result, the transmitting and receiving unit 11 generates and outputs a plurality of pieces of reflected-wave data (reception signals) for each of the scanning lines. Further, the B-mode processing unit 13 extracts the harmonic component by performing an addition/subtraction process according to the modulation method on the plurality of pieces of reflected-wave data (the reception signals) corresponding to each of the scanning lines. After that, the B-mode processing unit 13 generates B-mode data by performing an envelope detection process or the like on the reflected-wave data (the reception signals) of the harmonic component.

For example, when implementing the PM method, the transmitting and receiving unit 11 transmits, according to a scan sequence set by the controlling unit 18, an ultrasound wave twice for each of the scanning lines, the ultrasound waves in the two transmissions having the same amplitude and opposite phase polarities such as (−1, 1). After that, the transmitting and receiving unit 11 generates a reception signal from the “−1” transmission and a reception signal from the “1” transmission, so that the B-mode processing unit 13 adds these two reception signals together. As a result, it is possible to generate a signal from which the fundamental wave component is eliminated and in which the second harmonic component chiefly remains. After that, the B-mode processing unit 13 performs an envelope detection process or the like on the signal and generates THI B-mode data or CHI B-mode data.

As another example, to perform the THI process, a method has been in practical use by which an imaging process is performed by using a second harmonic component contained in the reception signals and a difference tone component. According to an imaging method using a difference tone component, for example, the ultrasound probe 1 is caused to transmit a transmission ultrasound wave having a synthesized waveform obtained by synthesizing a first fundamental wave of which the center frequency is “f1” with a second fundamental wave of which the center frequency is “f2” that is higher than “f1”. The synthesized waveform is a waveform obtained by synthesizing the waveform of the first fundamental wave with the waveform of the second fundamental wave, both of which have the phases thereof adjusted, so that a difference tone component having the same polarity as the second harmonic component occurs. The transmitting and receiving unit 11 transmits the transmission ultrasound wave having the synthesized waveform twice, for example, while inverting the phase thereof. In that situation, for example, by adding the two reception signals together, the B-mode processing unit 13 extracts the harmonic component from which the fundamental wave component is eliminated and in which the difference tone component and the second harmonic component chiefly remain, before performing an envelope detection process or the like.

Returning to the description of FIG. 1, the Doppler processing unit 14 generates data (Doppler data) by extracting motion information based on the Doppler effect of a moving object that is present in the scanned region, by performing a frequency analysis on the reflected-wave data read from the buffer 12. More specifically, as the motion information of the moving object, the Doppler processing unit 14 generates the Doppler data by extracting an average velocity, a dispersion value, a power value, and the like with respect to multiple points. In this situation, examples of the moving object include bloodstream, a tissue such as the cardiac wall, and a contrast agent.

By using the function of the Doppler processing unit 14 capable of extracting the motion information of the moving object, the ultrasound diagnosis apparatus according to the first embodiment is able to implement a color Doppler method which may be called a Color Flow Mapping (CFM) method or a Tissue Doppler Imaging (TDI) method. Further, by using the function of the Doppler processing unit 14, the ultrasound diagnosis apparatus according to the first embodiment is also able to perform an elastography process. In a color Doppler mode, the Doppler processing unit 14 generates, as the motion information of the moving object, color Doppler data by extracting an average velocity, a dispersion value, a power value, and the like with respect to multiple points in a two-dimensional space or a three-dimensional space.

In a tissue Doppler mode, the Doppler processing unit 14 generates, as the motion information of the tissue serving as the moving object, tissue Doppler data by extracting an average velocity, a dispersion value, a power value, and the like with respect to multiple points in a two-dimensional space or a three-dimensional space. In an elastography mode, the Doppler processing unit 14 calculates a displacement by time-integrating velocity distribution information obtained from the tissue Doppler data. Further, by performing a predetermined calculation (e.g., a spatial differential) on the calculated displacement, the Doppler processing unit 14 calculates local strains in the tissue. Further, by color coding values expressing the local strains in the tissue, the Doppler processing unit 14 generates strain distribution information. The harder a tissue is, the lower is the tendency for the tissue to change the form thereof. Consequently, the strain value of a harder tissue is smaller, whereas the strain value of a softer tissue in the subject's body is larger. In other words, the strain value is a value that indicates the hardness (the elasticity) of the tissue. In the elastography mode, for example, the tissue is caused to change the form thereof, when the operator manually vibrates the ultrasound probe 1 abutting against the body surface of the subject so as to apply and release pressure to and from the tissue. Alternatively, in the elastography mode, for example, the tissue is caused to change the form thereof, when a force is applied thereto with acoustic emission pressure.

In this situation, the B-mode processing unit 13 and the Doppler processing unit 14 shown in FIG. 1 are able to process both two-dimensional reflected-wave data and three-dimensional reflected-wave data. In other words, the B-mode processing unit 13 is able to generate two-dimensional B-mode data from the two-dimensional reflected-wave data and to generate three-dimensional B-mode data from the three-dimensional reflected-wave data. The Doppler processing unit 14 is able to generate two-dimensional Doppler data from the two-dimensional reflected-wave data and to generate three-dimensional Doppler data from the three-dimensional reflected-wave data. Ultrasound scans performed in the Doppler mode or the elastography mode as well as processes performed by the Doppler processing unit 14 in the first embodiment will be explained later in detail.

The image generating unit 15 generates ultrasound image data from the data generated by the B-mode processing unit 13 and the Doppler processing unit 14. From the two-dimensional B-mode data generated by the B-mode processing unit 13, the image generating unit 15 generates two-dimensional B-mode image data in which the strength of the reflected wave is expressed by a degree of brightness. Further, from the two-dimensional Doppler data generated by the Doppler processing unit 14, the image generating unit 15 generates two-dimensional Doppler image data expressing moving object information. The two-dimensional Doppler image data is velocity image data, dispersion image data, power image data, or image data combining any of these types of image data.

In this situation, generally speaking, the image generating unit 15 converts (by performing a scan convert process) a scanning line signal sequence from an ultrasound scan into a scanning line signal sequence in a video format used by, for example, television and generates display-purpose ultrasound image data. More specifically, the image generating unit 15 generates the display-purpose ultrasound image data by performing a coordinate transformation process compliant with the ultrasound scanning mode used by the ultrasound probe 1. Further, as various types of image processing processes other than the scan convert process, the image generating unit 15 performs, for example, an image processing process (a smoothing process) to re-generate a luminance-average image or an image processing process (an edge enhancement process) using a differential filter within images, while using a plurality of image frames obtained after the scan convert process is performed. Further, the image generating unit 15 synthesizes text information of various parameters, scale graduations, body marks, and the like with the ultrasound image data.

In other words, the B-mode data and the Doppler data are the ultrasound image data before the scan convert process is performed. The data generated by the image generating unit 15 is the display-purpose ultrasound image data obtained after the scan convert process is performed. The B-mode data and the Doppler data may also be referred to as raw data. The image generating unit 15 generates display-purpose two-dimensional ultrasound image data, from the two-dimensional ultrasound image data before the scan convert process is performed.

Further, the image generating unit 15 generates three-dimensional B-mode image data by performing a coordinate transformation process on the three-dimensional B-mode data generated by the B-mode processing unit 13. Further, the image generating unit 15 generates three-dimensional Doppler image data by performing a coordinate transformation process on the three-dimensional Doppler data generated by the Doppler processing unit 14. The image generating unit 15 generates “the three-dimensional B-mode image data or the three-dimensional Doppler image data” as “three-dimensional ultrasound image data (volume data)”.

Further, the image generating unit 15 performs a rendering process on the volume data, to generate various types of two-dimensional image data used for displaying the volume data on the monitor 2. Examples of the rendering process performed by the image generating unit 15 include a process to generate Multi Planar Reconstruction (MPR) image data from the volume data by implementing an MPR method. Another example of the rendering process performed by the image generating unit 15 is Volume Rendering (VR) process to generate two-dimensional image data that reflects three-dimensional information.

The image memory 16 is a memory for storing therein the display-purpose image data generated by the image generating unit 15. Further, the image memory 16 is also able to store therein the data generated by the B-mode processing unit 13 and the Doppler processing unit 14. After a diagnosis process, for example, the operator is able to invoke the B-mode data or the Doppler data stored in the image memory 16. The invoked data serves as the display-purpose ultrasound image data via the image generating unit 15. Further, the image memory 16 is also able to store therein the reflected-wave data output by the transmitting and receiving unit 11.

The internal storage unit 17 stores therein various types of data such as a control computer program (hereinafter, “control program”) to realize ultrasound transmissions and receptions, image processing, and display processing, as well as diagnosis information (e.g., patients' IDs, medical doctors' observations), diagnosis protocols, and various types of body marks. Further, the internal storage unit 17 may be used, as necessary, for storing therein any of the image data stored in the image memory 16. Further, it is possible to transfer the data stored in the internal storage unit 17 to external apparatuses via an interface (not shown). Further, the internal storage unit 17 is also able to store therein data that is transferred thereto from an external apparatus via an interface (not shown).

The controlling unit 18 is configured to control the entire processes performed by the ultrasound diagnosis apparatus. More specifically, based on the various types of setting requests input by the operator via the input device 3 and various types of control programs and various types of data read from the internal storage unit 17, the controlling unit 18 controls processes performed by the transmitting and receiving unit 11, the B-mode processing unit 13, the Doppler processing unit 14, and the image generating unit 15. Further, the controlling unit 18 exercises control so that the monitor 2 displays the display-purpose ultrasound image data stored in the image memory 16 and the internal storage unit 17.

The transmitting and receiving unit 11 and the like installed in the apparatus main body 10 may be configured with hardware such as an integrated circuit or may be configured with a computer program that is structured with modules in the manner of software.

An overall configuration of the ultrasound diagnosis apparatus according to the first embodiment has thus been explained. The ultrasound diagnosis apparatus according to the first embodiment configured as described above displays, at the same time, B-mode image data that is tissue image data and color Doppler image data that is bloodstream image data. To realize the display, the controlling unit 18 causes the ultrasound probe 1 to perform a first ultrasound scan to obtain information related to motion of a moving object within a first scanned region. The first ultrasound scan is, for example, an ultrasound scan to acquire color Doppler image data in the color Doppler mode. Further, together with the first ultrasound scan, the controlling unit 18 causes the ultrasound probe 1 to perform a second ultrasound scan to obtain information about a tissue form within a second scanned region. The second ultrasound scan is, for example, an ultrasound scan to acquire B-mode image data in the B-mode.

By controlling the ultrasound probe 1 via the transmitting and receiving unit 11, the controlling unit 18 causes the first ultrasound scan and the second ultrasound scan to be performed. The first scanned region and the second scanned region may be the same region as each other. Alternatively, the first scanned region may be smaller than the second scanned region. Conversely, the second scanned region may be smaller than the first scanned region.

According to a commonly-used color Doppler method, an ultrasound wave is transmitted multiple times in mutually the same direction, so that motion information of bloodstream is extracted by performing a frequency analysis based on the Doppler effect on the signals received as a result of the transmissions. A data sequence of reflected-wave signals from mutually the same location in the data obtained by transmitting an ultrasound wave multiple times in mutually the same direction will be referred to as a “packet”. According to the commonly-used color Doppler method, the packet size ranges from 5 to 16, approximately. The signals from the bloodstream are extracted by applying a wall filter (as well as known MTI filter) to the packet, the wall filter being configured to suppress signals from the tissue (which may be referred to as “clutter signals”). Further, according to the commonly-used color Doppler method, bloodstream information such as an average velocity, a dispersion, a power, and/or the like is displayed based on the extracted signals.

The commonly-used color Doppler method, however, has problems that can be explained as follows: Because packets are closed in ultrasound scan frames according to the commonly-used color Doppler method, when the packet size is arranged to be larger, the frame rate drops. Also, according to the commonly-used color Doppler method, an Infinite Impulse Response (IIR) filter is often used as the wall filter. When the packet size is small, because the IIR filter has a transient response, the level of performance of the IIR filter is degraded. The IIR filter is a type of Moving Target Indicator (MTI) filter, which is a High Pass Filter (HPF).

To solve the problems described above, a high frame rate method will be used, by which the motion information of a moving object such as the bloodstream is imaged at a high frame rate. According to the high frame rate method, instead of handling the packets as being closed in the frames, signals from mutually the same location among mutually-different frames are handled as a packet. According to the high frame rate method, an ultrasound scan that is similar to a B-mode scan is performed. In other words, according to the high frame rate method, an ultrasound transmission/reception is performed once for each of a plurality of scanning lines structuring a scanned region corresponding to one frame. Further, according to the high frame rate method, data processing is performed along the frame direction on a sequence of pieces of data (hereinafter, a “data sequence”) that are in mutually the same position among the mutually-different frames.

As a result, by using the high frame rate method, it is possible to arrange the wall filtering process to be a process performed on data having an infinite length, instead of a process performed on the packets, which is data having a finite length. It is therefore possible to enhance the level of performance of the IIR filter and, at the same time, it is possible to display the bloodstream information at the same frame rate as a scan frame rate.

In other words, according to the high frame rate method, because the Pulse Repetition Frequency (PRF) is equal to the frame rate, the aliasing velocity becomes low. Thus, the high frame rate method has an advantage where it is possible to observe the bloodstream even at a low flow rate.

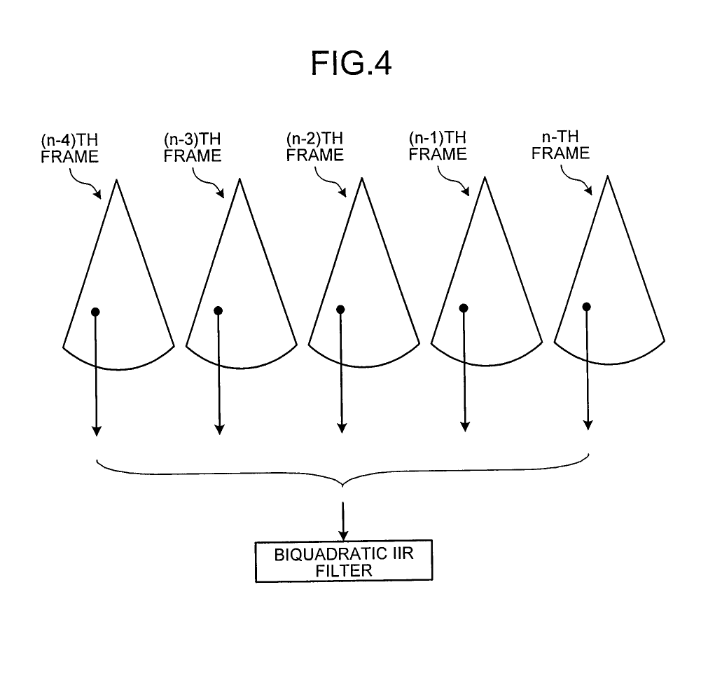

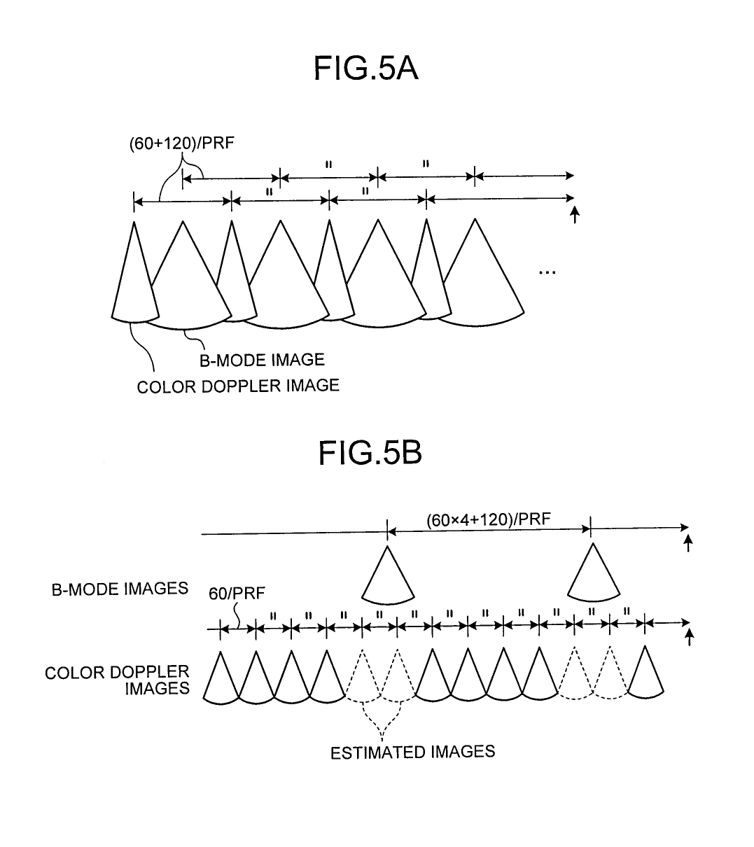

The Doppler processing unit 14 according to the first embodiment is able to implement the high frame rate method, together with the commonly-used color Doppler method. In the following sections, the Doppler processing unit 14 will be explained, with reference to FIGS. 3 and 4. FIG. 3 is a block diagram of an exemplary configuration of the Doppler processing unit shown in FIG. 1. FIG. 4 is a drawing for explaining a wall filtering process performed by implementing the high frame rate method.

As illustrated in FIG. 3, the Doppler processing unit 14 includes a wall filter 141, an auto-correlation calculating unit 142, an average velocity/dispersion calculating unit 143, a power calculating unit 144, a power adding unit 145, and a logarithmic compression unit 146. Further, as illustrated in FIG. 3, the Doppler processing unit 14 includes an average power calculating unit 147 and a power correcting unit 148.

The wall filter 141 is a processing unit configured to perform an IIR filtering process and is configured by using a biquadratic IIR filter, for example. As illustrated in FIG. 4, to obtain IIR filter output data (a bloodstream signal) for an “n-th” frame, the wall filter 141 uses reflected-wave data (a reception signal) in the “n-th” frame, reflected-wave data (reception signals) in the past four frames (namely, the “(n−4)th” to the “(n−1)th” frames), and IIR filter output data (bloodstream signals) in the past four frames, corresponding to mutually the same position. Each of these pieces of reflected-wave data is, as described above, reflected-wave data that is generated by performing an ultrasound transmission/reception once for each of the plurality of scanning lines structuring the scanned region (the first scanned region) corresponding to one frame. As a result of the IIR filtering process performed by the wall filter 141, it is possible to extract a bloodstream signal from which clutter signals are eliminated, with a high level of precision. When an ultrasound scan is performed using the high frame rate method, because the data having an infinite length is continuously input to the wall filter 141, no transient response occurs during the wall filtering process.

Returning to the description of FIG. 3, the auto-correlation calculating unit 142 calculates an auto-correlation value by calculating complex conjugates between the IQ signals of the bloodstream signal in the latest frame and the IQ signals of the bloodstream signal in the immediately preceding frame. The average velocity/dispersion calculating unit 143 calculates an average velocity and a dispersion from the auto-correlation value calculated by the auto-correlation calculating unit 142.

Further, the power calculating unit 144 calculates a power value by adding together a value obtained by raising the absolute value of the real-part of the IQ signals of the bloodstream signal to the second power and another value obtained by raising the absolute value of the imaginary-part to the second power. The power value is a value indicating the magnitude of scattering caused by reflecting objects (e.g., blood cells) that are smaller than the wavelength of the transmitted ultrasound wave. The power adding unit 145 adds together the power values at each of the points among arbitrary frames. The logarithmic compression unit 146 performs a logarithmic compression on the output from the power adding unit 145. The pieces of data output by the average velocity/dispersion calculating unit 143 and the logarithmic compression unit 146 are output to the image generating unit 15 as Doppler data. The Doppler processing unit 14 is able to implement both the high frame rate method and the commonly-used color Doppler method. Further, in addition to the motion information of the bloodstream, the Doppler processing unit 14 is also able to generate motion information of a tissue.

According to the high frame rate method described above, however, it is easier for clutter signals to pass the wall filter, and a motion artifact may occur in some situations. In particular, while the ultrasound probe 1 is being moved, the entire screen is displayed with clutter signals. In addition, when an ultrasound scan is performed by implementing the commonly-used color Doppler method described above, a motion artifact occurs when the aliasing velocity is lowered.

To solve these problems, the Doppler processing unit 14 includes the average power calculating unit 147 and the power correcting unit 148. The average power calculating unit 147 is configured to calculate an average power value for one frame or within a local region, on the basis of a power addition value on which a logarithmic compression has been performed. The power correcting unit 148 is configured to perform a correcting process on each of such points (pixels) of which the average power value exceeds a threshold value. More specifically, the power correcting unit 148 subtracts “a value obtained by multiplying a difference between the average power value and the threshold value by a predetermined coefficient” from the power value of each of such pixels of which the average power value exceeds the threshold value. With this arrangement, the power correcting unit 148 corrects the power value of each of such pixels of which the average power value exceeds the threshold value.

The operator is able to arrange a setting as to whether the power correcting process is to be performed or not. While the power correcting process is performed, the data output by the power correcting unit 148 is also output to the image generating unit 15 as Doppler data. While the power correcting process is performed, the image generating unit 15 generates, for example, bloodstream image data rendering information about the power and a direction (the sign of the velocity). It should be noted that the first embodiment is also applicable to a situation where the power correcting process is not performed.

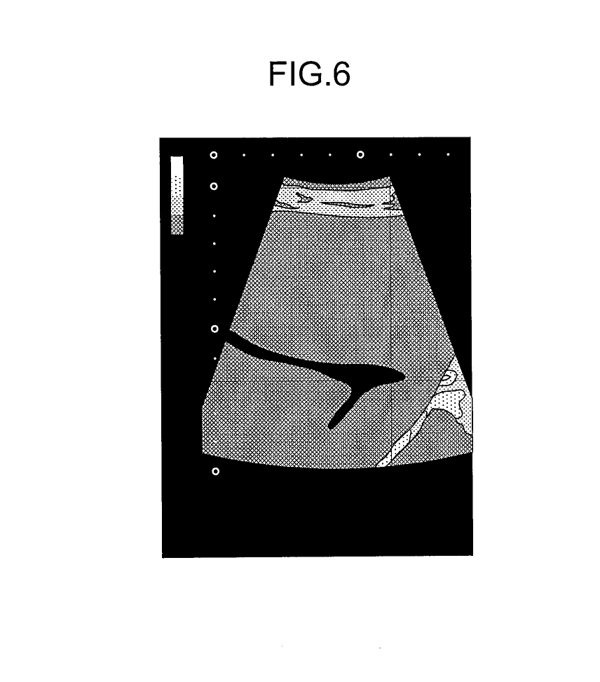

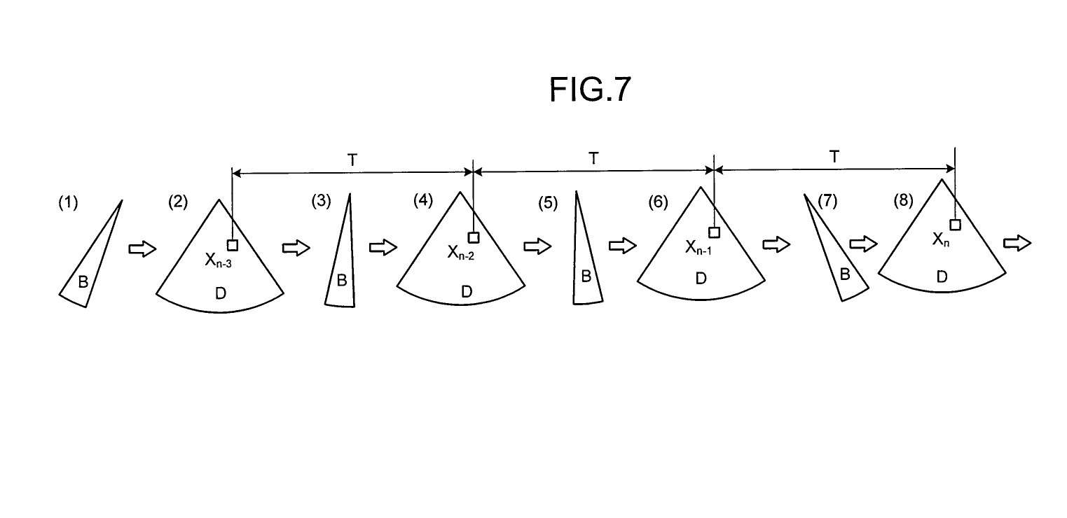

Next, three conventional methods that can be used for displaying the tissue image data and the bloodstream image data at the same time will be explained as examples. These three methods described below, however, have various problems. The problems will be explained with reference to FIGS. 4, 5A, 5B, and 6. FIGS. 5A and 5B are drawings for explaining the examples of the conventional methods. FIG. 6 is a drawing of an example of a problem with the conventional methods.

A first method is the high frame rate method by which, as explained with reference to FIG. 4, an ultrasound transmission/reception is performed once for each of the plurality of scanning lines structuring the scanned region corresponding to one frame, so that the bloodstream signal and the tissue signal are extracted and become subject to an imaging process while using the same piece of reflected-wave data. In other words, according to the first method, the first ultrasound scan is the same as the second ultrasound scan.

The first method, however, has the following three problems: A first problem of the first method is caused by the fact that it is necessary to raise the gain of a pre-amplifying process performed by the amplifying circuit in the transmitting and receiving unit 11, for the purpose of obtaining the bloodstream signal with an excellent sensitivity. In other words, when the gain is raised, the reflected-wave signals from a tissue having a high reflection intensity are more prone to saturate in the processes at subsequent stages. If the saturation has occurred, the gray-scale levels of the tissue having the high reflection intensity become lower, and obtained B-mode image data has less contrast.

A second problem of the first method is caused by the fact that the frame rate according to the first method is a PRF. In other words, it is necessary to raise the frame rate to reduce aliasing of the bloodstream velocity. However, if the raster density is lowered for the purpose of raising the frame rate, the resolution of the B-mode image data in the azimuth direction is degraded. As a result, the B-mode image displayed on the monitor 2 will be an image experiencing a significant cross-flow and thus having a lower image quality, as illustrated in FIG. 6.

A third problem of the first method is that it is required to perform fundamental-wave transmissions/receptions in order to obtain the bloodstream signal with an excellent sensitivity. For this reason, it is not possible to generate and display B-mode image data by performing the THI process realized by receiving a second harmonic, which is a popularly-used method for observing tissues in recent years.

According to a second method for displaying the tissue image data and the bloodstream image data at the same time, the second ultrasound scan to acquire the tissue image data (the B-mode images) and the first ultrasound scan to acquire the bloodstream image data (the color Doppler images) are performed separately and alternately, as illustrated in FIG. 5A. In an ultrasound scan procedure illustrated in FIG. 5A, the first scanned region for a color Doppler purpose is structured with “60” scanning lines, whereas the second scanned region for a B-mode purpose is structured with “120” scanning lines. In the example in FIG. 5A, the first ultrasound scan and the second ultrasound scan are performed so that an ultrasound scan for each of the scanning lines is performed at regular interval equal to “1/PRF”. In the example in FIG. 5A, the frame time period is calculated as “(60+120)/PRF”, which is a sum of the time period “60/PRF” required by the first ultrasound scan corresponding to one frame and the time period “120/PRF” required by the second ultrasound scan corresponding to one frame.

According to the second method, although it is possible to acquire B-mode image data having high image quality, a problem remains where the velocity is prone to experience aliasing due to a fall in the frame rate of the bloodstream image data.

According to a third method for displaying the tissue image data and the bloodstream image data at the same time, as illustrated in FIG. 5B, the first ultrasound scan to acquire the bloodstream image data (the color Doppler images) is performed constantly, whereas the second ultrasound scan to acquire the tissue image data (the B-mode images) is inserted once in every predetermined time period. Further, according to the third method, a bloodstream image signal during a time period when the second ultrasound scan is performed is estimated by performing an interpolation process based on the bloodstream signals before and after the time period when the second ultrasound scan is performed, so that estimated images can be displayed. In the example illustrated in FIG. 5B, the frame time period of the color Doppler images including the estimated images is “60/PRF”, whereas the frame time period of the B-mode images is “(60×4+120)/PRF”.

However, because the wall filter is a high pass filter, a problem remains where using the estimated signals leads to an occurrence of noise, and the bloodstream image data thus contains the noise. In addition, because the wall filter is an IIR filter, the impact of the noise is spread to a certain number of frames before and after the estimated frames. As a result, the images contain a large amount of noise as a whole.

As explained above, according to the first, the second, and the third methods, the image quality of the images indicating the moving object information and the tissue images that are displayed at the same time may be degraded in some situations. To cope with these situations, the controlling unit 18 according to the first embodiment causes the second ultrasound scan to be performed in the manner described below, for the purpose of improving the image quality of the images indicating the moving object information and the tissue images that are displayed at the same time.

Specifically, as the second ultrasound scan, the controlling unit 18 according to the first embodiment causes the ultrasound probe 1 to perform an ultrasound scan in each of a plurality of sectioned regions into which the second scanned region is divided, in a time-division manner between the first ultrasound scans. In other words, according to the first embodiment, a part of the second ultrasound scan is performed between the first ultrasound scans, so that the second ultrasound scan corresponding to one frame is completed in a time period during which the first ultrasound scans corresponding to a certain number of frames are performed. With this arrangement, according to the first embodiment, it is possible to set ultrasound transmission/reception conditions for the first ultrasound scan and for the second ultrasound scan, independently of each other.

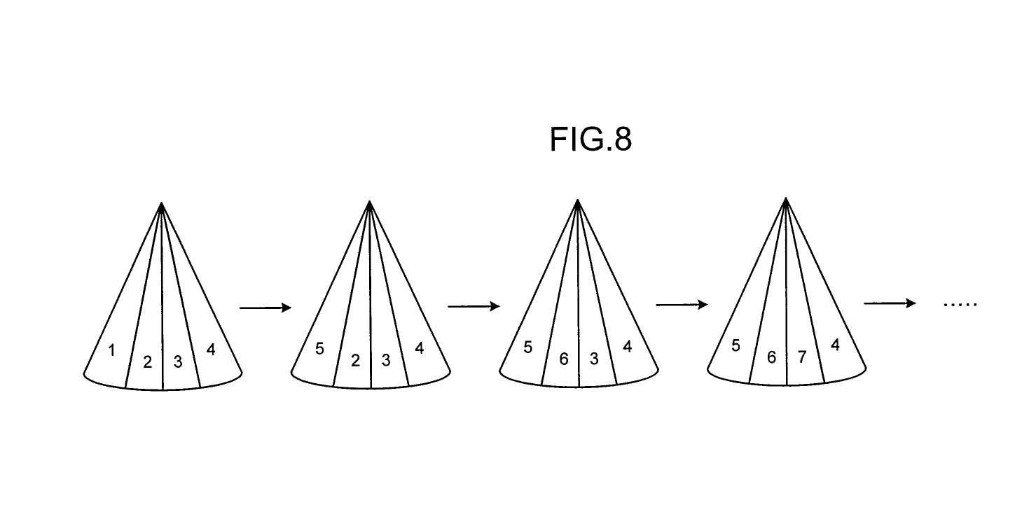

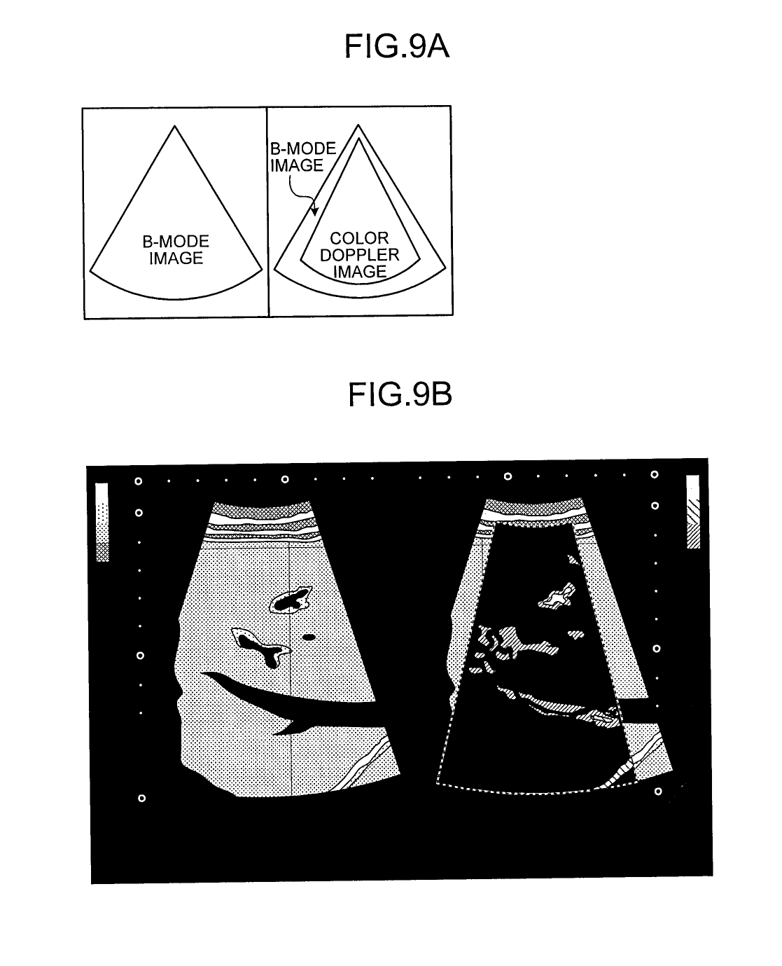

An example of the controlling process described above will be explained, with reference to FIGS. 7 and 8. FIGS. 7 and 8 are drawings for explaining the controlling unit according to the first embodiment. For example, on the basis of an instruction from the operator or information in an initial setting or the like, the controlling unit 18 divides the second scanned region into four sectioned regions (first to fourth sectioned regions). The regions each marked with a “B” in FIG. 7 are the regions in which an ultrasound scan is performed by using a B-mode transmission/reception condition. The regions each marked with a “D” in FIG. 7 are the regions in which an ultrasound scan is performed by using a color Doppler mode transmission/reception condition. For example, the regions each marked with a “D” in FIG. 7 are the regions in which the ultrasound scan is performed by implementing the high frame rate method described above. In other words, the first ultrasound scan illustrated in FIG. 7 is performed by performing an ultrasound transmission/reception once for each of the scanning lines, unlike in the commonly-used color Doppler method by which an ultrasound wave is transmitted multiple times in mutually the same direction so as to receive the reflected wave multiple times. In other words, the controlling unit 18 causes the ultrasound scan to acquire bloodstream Doppler image data to be performed, as the first ultrasound scan. Further, as the first ultrasound scan, the controlling unit 18 causes the ultrasound scan to be performed according to a method for obtaining information related to motion of the moving object by which a high pass filtering process (e.g., the IIR filtering process) is performed along the frame direction on the reception signals (the reflected-wave data) obtained from the plurality of scanning lines structuring the first scanned region. As the first ultrasound scan, the controlling unit 18 according to the first embodiment causes the ultrasound scan to be performed according to a method for obtaining a data sequence along the frame direction by which the reception signals are obtained from the plurality of scanning lines structuring the first scanned region by performing an ultrasound transmission/reception once for each of the scanning lines, so that the high pass filtering process is performed on the obtained reception signals. In other words, as the first ultrasound scan, the controlling unit 18 according to the first embodiment causes the ultrasound scan to be performed according to the method (the high frame rate method) for obtaining the information related to the motion of the moving object by using the reflected waves corresponding to the plurality of frames, by which an ultrasound transmission/reception is performed once each for the plurality of scanning lines structuring the first scanned region.

First, the controlling unit 18 causes an ultrasound scan for the first sectioned region to be performed as the second ultrasound scan (see FIG. 7(1)) and subsequently causes the first ultrasound scan for the second scanned region (corresponding to one frame) to be performed (see FIG. 7(2)). After that, the controlling unit 18 causes an ultrasound scan for the second sectioned region to be performed as the second ultrasound scan (see FIG. 7(3)) and subsequently causes the first ultrasound scan for the second scanned region (corresponding to one frame) to be performed (see FIG. 7(4)). After that, the controlling unit 18 causes an ultrasound scan for the third sectioned region to be performed as the second ultrasound scan (see FIG. 7(5)) and subsequently causes the first ultrasound scan for the second scanned region (corresponding to one frame) to be performed (see FIG. 7(6)). After that, the controlling unit 18 causes an ultrasound scan for the fourth sectioned region to be performed as the second ultrasound scan (see FIG. 7(7)) and subsequently causes the first ultrasound scan for the second scanned region (corresponding to one frame) to be performed (see FIG. 7(8)).

In this situation, as illustrated in FIG. 7, the controlling unit 18 arranges the first ultrasound scans to be performed at regular intervals. In other words, a “point X” on a “given scanning line” in the first scanned region is scanned once each during the first ultrasound scans at steps (2), (4), (6), and (8) in FIG. 7. At that time, control is exercised so that each of the scanning intervals has a constant value “T”. More specifically, the controlling unit 18 arranges the first ultrasound scans to be performed at the regular intervals, by arranging the time periods required by the sectioned scans of the second ultrasound scan to be equal to one another. For example, the controlling unit 18 exercises control so that the time periods required by the sectioned scans of the second ultrasound scan performed at steps (1), (3), (5), and (7) in FIG. 7 are always equal. The controlling unit 18 arranges the size of the sectioned regions into which the second scanned region is divided, the quantities of scanning lines, and the densities and the depths of the scanning lines to be equal. For example, if the quantities of scanning lines are equal, the time periods required by the sectioned scans of the second ultrasound scan are also equal. As illustrated in FIG. 7, the Doppler processing unit 14 outputs the motion information of the bloodstream at the “point X”, by performing the IIR filtering process described above on the data sequence (Xₙ₋₃, Xₙ₋₂, Xₙ₋₁, and Xₙ) marked with the “D” that are in mutually the same position and from the mutually-different frames.

As explained above, according to the first embodiment, because it is possible to set the ultrasound transmission/reception conditions for the first ultrasound scan and for the second ultrasound scan independently of each other, it is possible to solve the problems described above. First, because it is possible to optimize the gain of the pre-amplifying process separately for the first ultrasound scan and for the second ultrasound scan, it is possible to avoid the situation where the reflected-wave signals from the tissue saturate.

Further, because the second ultrasound scan is performed as the sectioned scans that are performed multiple times between the first ultrasound scans each corresponding to one frame, it is possible to lower the degree of the frame rate drop that is caused by performing the second ultrasound scan corresponding to one frame. As a result, it is possible to raise the aliasing velocity of the bloodstream.

Further, because the second ultrasound scan corresponding to one frame is performed in the form of the sectioned scans performed multiple times, it is possible to increase the density of the scanning lines in the B-mode, and it is therefore possible to, for example, avoid the situation where cross-flows occur in the B-mode image data.

Further, because it is possible to set the ultrasound transmission/reception conditions for the first ultrasound scan and for the second ultrasound scan independently of each other, it is possible to acquire the tissue image data by performing the THI process. In other words, it is possible to perform the second ultrasound scan under an ultrasound transmission/reception condition suitable for performing the THI process with the filtering process described above. Further, it is possible to perform the second ultrasound scan under an ultrasound transmission/reception condition suitable for performing the THI process according to an imaging method by which ultrasound transmissions having a plurality of rates are performed on any one scanning line, such as the AM method, the PM method, the AMPM method, or the method using difference tone component.

It should be noted that, however, according to the method in the first embodiment, the frame rate of the tissue images becomes low, as a trade-off. For example, in the example shown in FIG. 7, the pieces of bloodstream information each corresponding to one frame are output at the intervals “T”. In other words, the frame rate of the bloodstream images (the color Doppler images) is “1/T”. Further, in the example shown in FIG. 7, although the pieces of partial B-mode data (the tissue images) are also output at the intervals “T”, only “¼” of the entire second scanned region is scanned while the bloodstream images corresponding to one frame are output.

In other words, in the example shown in FIG. 7, the frame rate to complete the scanning of the entire second scanned region is “1/(4T)”. Further, when the THI process is performed according to the imaging method by which the ultrasound transmissions having a plurality of rates are performed on any one scanning line, the number of times the ultrasound transmission needs to be performed to obtain the reception signals corresponding to one frame increases. Thus, it is necessary to increase the quantity of sections into which the second scanned region is divided, in comparison to an ordinary B-mode image taking process and to a situation where the THI process is performed with the filtering process. For example, when using the PM method, the quantity of sections into which the second scanned region is divided is changed from four sections to eight sections. In that situation, the frame rate to complete the scanning of the entire second scanned region is “1/(8T)”. As explained here, when the method according to the first embodiment is used, the frame rate of the tissue images is slower than the frame rate of the bloodstream images. This is because the purpose of the ultrasound scans performed according to the present method is to raise the frame rate of the bloodstream images. In other words, the aliasing velocity of the bloodstream is determined by the frame rate “1/T” of the bloodstream images obtained by using the high frame rate method.

In this situation, as explained above, when the high frame rate method is used, because the PRF is equal to the frame rate, it is necessary to raise the scan rate “1/T” to view bloodstream having a high flow rate without aliasing. In other words, it is necessary to keep the value of “T” small. However, if the quantity of scanning lines for the tissue images and the bloodstream images to be eventually displayed is reduced for the purpose of keeping the value of “T” small, the image quality of the tissue images and the bloodstream images becomes low. For this reason, to maintain the image quality of the tissue images and the bloodstream images, it is desirable to reduce the quantity of scanning lines while maintaining the density of the scanning lines corresponding to the one-time sectioned scan in the B-mode. As a trade-off for performing such a process, the frame rate to display the complete tissue images becomes low, as mentioned above. However, generally speaking, when tissue images and bloodstream images are displayed at the same time, viewing the bloodstream is the main purpose, while the tissue images serve as a guide for viewing the bloodstream images. Thus, the problem caused by the frame rate of the tissue images becoming low is small.

However, it should be noted that, according to the first embodiment, when performing the second ultrasound scan illustrated in FIG. 7, the controlling unit 18 updates the tissue images for each of the sectioned scanned regions, instead of updating the tissue images at the intervals “4T”. This update control will be explained by using the second ultrasound scan illustrated in FIG. 7. As illustrated in FIG. 8, when B-mode image data in the first sectioned region is newly generated (see “5” in FIG. 8), while B-mode image data in the first to the fourth sectioned regions (see “1” to “4” in FIG. 8) is being displayed, the controlling unit 18 updates the B-mode image data “1” in the first sectioned region to “5”.

After that, as illustrated in FIG. 8, when B-mode image data in the second sectioned region is newly generated (see “6” in FIG. 8), the controlling unit 18 updates the B-mode image data “2” in the second sectioned region to “6”. Subsequently, as illustrated in FIG. 8, when B-mode image data in the third sectioned region is newly generated (see “7” in FIG. 8), the controlling unit 18 updates the B-mode image data “3” in the third sectioned region to “7”. After that, although not shown in the drawing, when B-mode image data in the fourth sectioned region is newly generated (“8”), the controlling unit 18 updates the B-mode image data “4” in the fourth sectioned region to “8”.

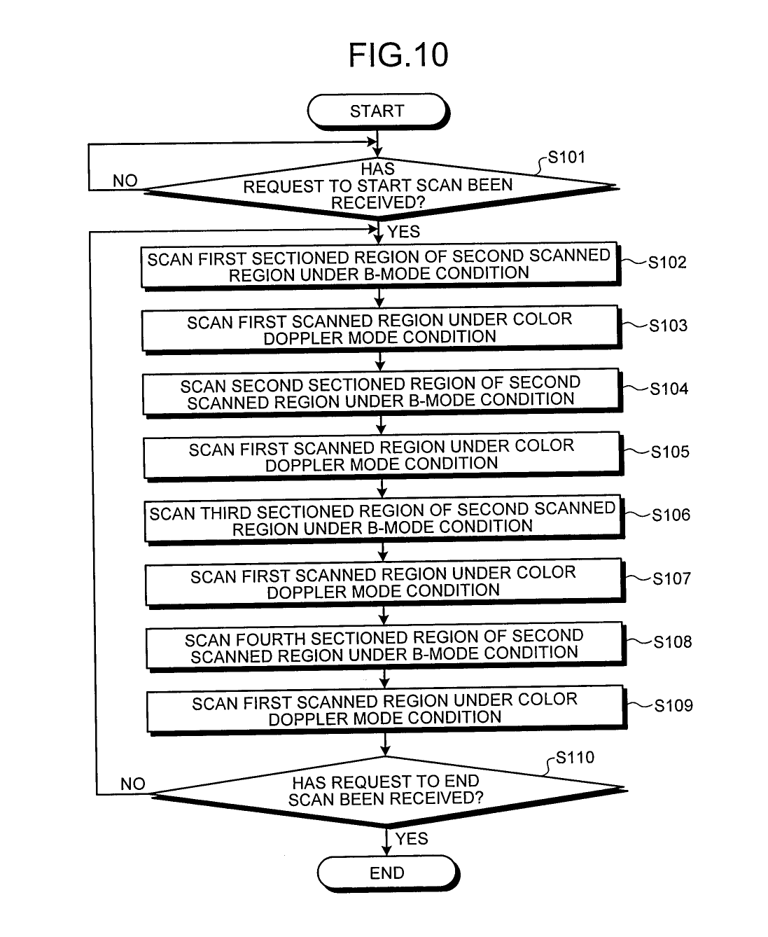

Subsequently, the controlling unit 18 exercises display control as shown in FIGS. 9A and 9B, for example. FIGS. 9A and 9B are drawings of examples of display modes according to the first embodiment. For example, under the control of the controlling unit 18, the monitor 2 displays the B-mode images (the tissue images) on the left side, while displaying superimposed images on the right side in which the B-mode images and the color Doppler images (the bloodstream images) are superimposed together, as shown in FIG. 9A. In the example shown in FIG. 9A, the first scanned region is set within the second scanned region.

FIG. 9B illustrates an example in which the B-mode images shown in FIG. 9A are “B-mode images generated by performing the THI process”, whereas the color Doppler images shown in FIG. 9A are power images. Alternatively, the B-mode images shown in FIG. 9A may be ordinary B-mode images. Further, the color Doppler images shown in FIG. 9A may be images in which velocity data and dispersion data are combined together. In another example, the images displayed on the right side of the monitor 2 may be the bloodstream images only. Further, if the power correcting process described above has been performed, the bloodstream images displayed on the right side of the monitor 2 may be bloodstream images in which information about both the power and the direction (the sign of the velocity) is rendered.

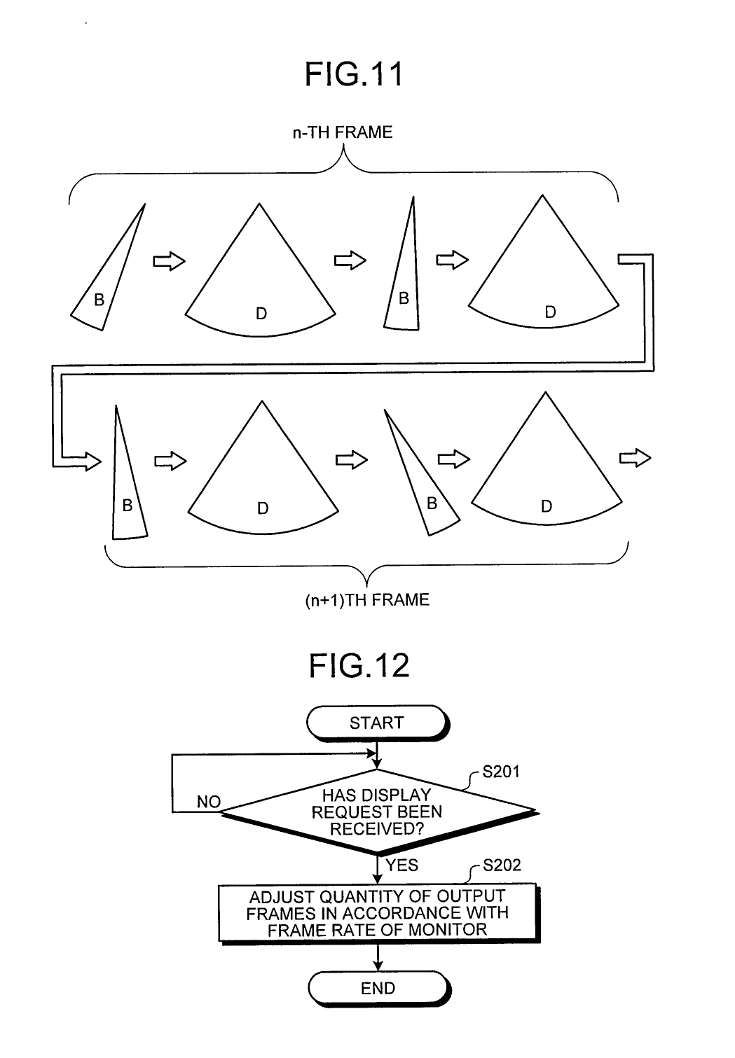

Next, an example of an ultrasound scan controlling process performed by the ultrasound diagnosis apparatus according to the first embodiment will be explained, with reference to FIG. 10. FIG. 10 is a flowchart for explaining an example of the ultrasound scan controlling process performed by the ultrasound diagnosis apparatus according to the first embodiment. FIG. 10 is a flowchart of an example in which the second scanned region is divided into four sections.

As shown in FIG. 10, the controlling unit 18 included in the ultrasound diagnosis apparatus according to the first embodiment judges whether a request to start an ultrasound scan has been received (step S101). If no request to start a scan has been received (step S101: No), the controlling unit 18 stands by until a request to start a scan is received.

On the contrary, if a request to start a scan has been received (step S101: Yes), the controlling unit 18 causes the first sectioned region of the second scanned region to be scanned under a B-mode condition (step S102) and subsequently, causes the first scanned region to be scanned under a color Doppler mode condition (step S103). After that, the controlling unit 18 causes the second sectioned region of the second scanned region to be scanned under the B-mode condition (step S104) and subsequently, causes the first scanned region to be scanned under the color Doppler mode condition (step S105).

After that, the controlling unit 18 causes the third sectioned region of the second scanned region to be scanned under the B-mode condition (step S106) and subsequently, causes the first scanned region to be scanned under the color Doppler mode condition (step S107). After that, the controlling unit 18 causes the fourth sectioned region of the second scanned region to be scanned under the B-mode condition (step S108) and subsequently, causes the first scanned region to be scanned under the color Doppler mode condition (step S109).

Further, the controlling unit 18 judges whether a request to end the ultrasound scan has been received (step S110). If no request to end the scan has been received (step S110: No), the process returns to step S102 where the controlling unit 18 causes the first sectioned region of the second scanned region to be scanned under the B-mode condition.

On the contrary, if a request to end the scan has been received (step S110: Yes), the controlling unit 18 ends the ultrasound scan controlling process. FIG. 10 describes the example in which the sectioned scan of the second ultrasound scan is performed first. However, the first embodiment may be configured so that the first ultrasound scan is performed first. Further, FIG. 10 describes the example in which it is judged whether a request to end the scan has been received at the point in time when all of the sectioned regions of the second scanned region have finished being processed. However, the first embodiment is also applicable to a situation where it is judged whether a request to end the scan has been received every time a scan for each of the sectioned regions of the second scanned region or a scan for the first scanned region is completed.

As explained above, according to the first embodiment, it is possible to set the ultrasound transmission/reception conditions for the first ultrasound scan and for the second ultrasound scan independently of each other, because the second ultrasound scan is performed in the form of the sectioned scans performed multiple times between the first ultrasound scans each corresponding to one frame. In other words, according to the first embodiment, it is possible to set an ultrasound transmission/reception condition optimal for the B-mode and to set an ultrasound transmission/reception condition optimal for the color Doppler mode. For example, according to the first embodiment, as the ultrasound transmission/reception condition for the second ultrasound scan, it is possible to set an ultrasound transmission/reception condition optimal for the THI process performed by implementing the PM method. As a result, according to the first embodiment, it is possible to improve the image quality of the bloodstream images (the images indicating the moving object information) and the tissue images that are displayed at the same time.

Further, according to the first embodiment, by arranging the first ultrasound scans to be performed at the regular intervals, it is possible to adjust the frame rate so that no aliasing occurs in the bloodstream images.

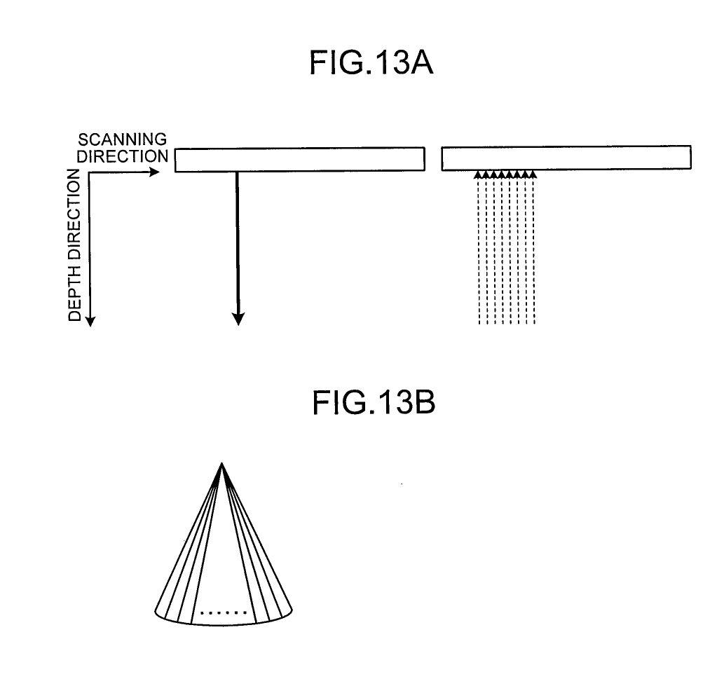

As a second embodiment, an example will be explained in which an output controlling process for generated image data is exercised by applying the scan control explained in the first embodiment, with reference to FIG. 11 and the like. FIG. 11 is a drawing for explaining the second embodiment.

An ultrasound diagnosis apparatus according to the second embodiment is configured to be similar to the ultrasound diagnosis apparatus according to the first embodiment explained with reference to FIG. 1. It should be noted, however, that the controlling unit 18 according to the second embodiment is configured to further exercise control so that the plurality of pieces of image data in the first scanned region that have been generated by the first ultrasound scan are output as one piece of image data, in accordance with the time period required by the first ultrasound scan performed at one time and the display frame rate of the monitor 2.

In the first embodiment, the bloodstream image data corresponding to one frame and the tissue image data updated by an amount corresponding to “1/the quantity of divided sections” are output every time the ultrasound scan in the color Doppler mode (i.e., the first ultrasound scan) and a sectioned scan of the B-mode ultrasound scan (a sectioned scan of the second ultrasound scan) is performed once. In this situation, if the generation frame rate of the bloodstream image data is higher than the display frame rate of the monitor 2, some of the frames are not displayed. For example, if the frame rate of the bloodstream images is 120 fps, it is possible to display only “½” of the image data output from the image generating unit 15, on the monitor 2 that is TV-scanned at 60 fps. In another example, if the frame rate of the bloodstream images is 1800 fps, it is possible to display only “ 1/30” of the image data output from the image generating unit 15 on the monitor 2.

The ultrasound diagnosis apparatus is configured so that, when the operator has pressed a freeze button included in the input device 3, all the frames stored in the image memory 16 are played back slowly, so that even the frames that are not displayable during a real-time display are displayed on the monitor 2. However, as for the bloodstream in the abdomen or the like having a low flow rate, even if bloodstream information at 60 fps or higher is output to be played back slowly, displayed images are almost the same. It is therefore not possible to provide the viewer with meaningful information. In fact, when the operator chooses to have a “cine” playback after the freeze, the operator needs to perform a frame-by-frame advancing operation on a large number of frames by using the trackball, which places a burden on the operator.

To cope with this situation, according to the second embodiment, the controlling unit 18 outputs, to the monitor 2 or the image memory 16, M pieces of bloodstream image data generated by repeating M times a pair made up of “B” and “D” illustrated in FIG. 7, as image data corresponding to one frame. The value “M” is calculated by the controlling unit 18, for example. In the example shown in FIG. 11, because “M=2” is satisfied, the controlling unit 18 either outputs one of the two pieces of bloodstream image data or outputs one averaging image data of the two pieces of bloodstream image data, as bloodstream image data for the “n-th” frame or the “(n+1)th” frame.

In the second embodiment also, the first ultrasound scan is performed as the first ultrasound scan according to the high frame rate method explained in the first embodiment. In that situation, although the display frame rate is “1/(M×T)”, the PRF remains at “1/T”.

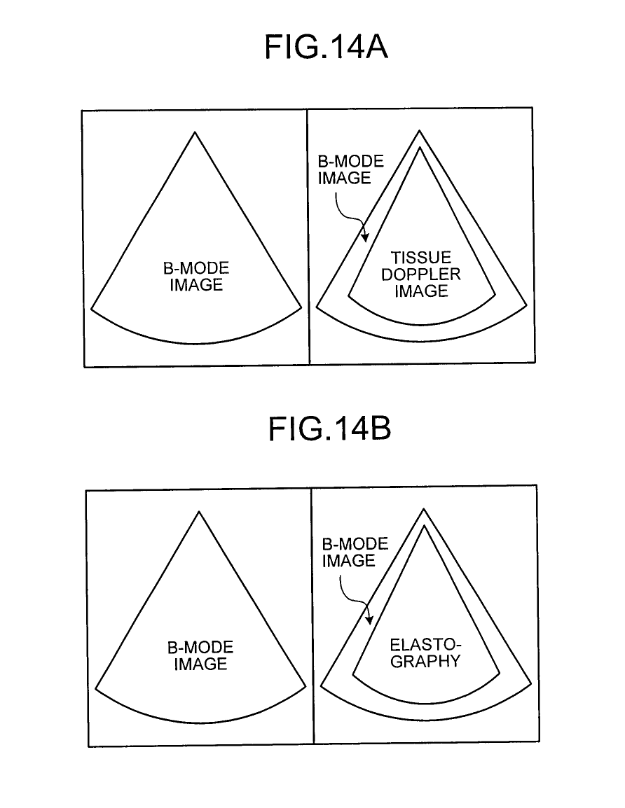

Next, an example of the output controlling process performed by the ultrasound diagnosis apparatus according to the second embodiment will be explained, with reference to FIG. 12. FIG. 12 is a flowchart for explaining the example of the output controlling process performed by the ultrasound diagnosis apparatus according to the second embodiment. FIG. 12 illustrates an example in which, when a playback display is realized after a freeze, the frame rate of the output to the monitor 2 is adjusted.

As shown in FIG. 12, the controlling unit 18 included in the ultrasound diagnosis apparatus according to the second embodiment judges whether a request to display the image data stored in the image memory 16 has been received (step S201). If no display request has been received (step S201: No), the controlling unit 18 stands by until a display request is received.

On the contrary, if a display request has been received (step S201: Yes), the controlling unit 18 adjusts the quantity of output frames in accordance with the frame rate of the first ultrasound scan and the display frame rate of the monitor 2 (step S202), and ends the process. As mentioned above, it is also possible to configure the second embodiment so that the quantity of output frames is adjusted when the image data is stored into the image memory 16.

As explained above, according to the second embodiment, the quantity of output frames that are output for the storing purpose or the quantity of output frames that are output for the display purpose is adjusted in accordance with the frame rate of the first ultrasound scan and the display frame rate of the monitor 2. More specifically, according to the second embodiment, the output frame rate of the bloodstream images is adjusted so as to be equal to or lower than the display frame rate of the monitor 2. With this arrangement, according to the second embodiment, it is possible to realize the frame-by-frame advancing operation during the “cine” playback without creating uncomfortable feelings for the viewer, by keeping the quantity of pieces of output data small, for the bloodstream information having a low flow rate, for example. In the example described above, the control is exercised so that the display frame rate “1/(M×T)” is equal to or lower than the frame rate (60 fps) of the monitor. Alternatively, as a method for determining the value “M” indicating the number of times of repetitions, it is also acceptable to arrange the frame rate to be equal to or lower than an arbitrary frame rate that is set in advance.

In the first and the second embodiments, the example is explained in which the two-dimensional tissue images and the two-dimensional bloodstream images are displayed by performing the two-dimensional scans. However, the first and the second embodiments are also applicable to a situation where three-dimensional tissue image data and three-dimensional bloodstream image data are generated by performing three-dimensional scans, so as to display MPR images and volume rendering images from these pieces of volume data.

More specifically, according to a third embodiment, the “Ds” in FIGS. 7 and 11 each represent the first ultrasound scan corresponding to one volume, whereas the “Bs” in FIGS. 7 and 11 each represent a sectioned scan of the second ultrasound scan corresponding to a sectioned volume. The bloodstream information in the “Ds” in FIGS. 7 and 11 is processed with respect to the sequence of pieces of data that are in mutually the same position among the mutually-different pieces of volume data.

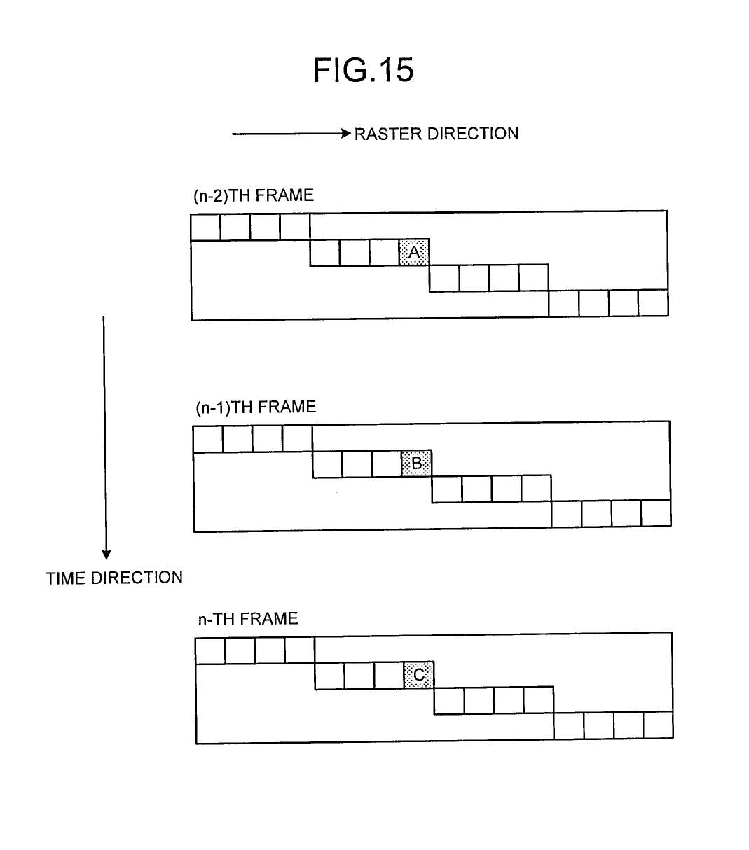

In the third embodiment, however, the volume rate corresponds to the PRF of the color Doppler images. For this reason, to raise the volume rate, the controlling unit 18 exercises control as shown in FIGS. 13A and 13B, for example. FIGS. 13A and 13B are drawings for explaining the third embodiment.

For example, as illustrated in FIG. 13A, the controlling unit 18 causes a parallel simultaneous reception to be performed for the purpose of raising the volume rate. FIG. 13A illustrates an example in which an 8-beam parallel simultaneous reception is performed. In FIG. 13A, the central axis of a transmitted ultrasound wave in the depth direction is indicated with a solid arrow, whereas the eight reflected-wave beams that are simultaneously received at the first time are indicated with dotted-line arrows. In a one-time ultrasound transmission/reception, the transmitting and receiving unit 11 receives reflected-wave signals on eight scanning lines from the ultrasound probe 1. As a result, the transmitting and receiving unit 11 is able to generate reflected-wave data on the eight scanning lines as a result of the one-time ultrasound transmission/reception. The quantity of beams in the parallel simultaneous reception (hereinafter, the “parallel simultaneous reception number”) can be set to an arbitrary value, in accordance with a required volume rate so as not to exceed an upper-limit quantity of beams which the transmitting and receiving unit 11 is able to simultaneously receive in parallel.

Further, as illustrated in FIG. 13B, for example, to raise the volume rate, the controlling unit 18 may reduce the quantity of scanning lines used in a one-time sectioned scan, by increasing the quantity of sections.

To raise the volume rate, the controlling unit 18 may implement both the parallel simultaneous reception and the increase of the quantity of sections. Further, to raise the volume rate, the controlling unit 18 may implement the parallel simultaneous reception in the first ultrasound scan, or may implement the parallel simultaneous reception in the second ultrasound scan, or may implement the parallel simultaneous reception in both of the first and the second ultrasound scans. The second ultrasound scan performed as a three-dimensional scan is, for example, an ultrasound scan for the THI process according to the AM method, the PM method, or the like.

According to the third embodiment, even if the scans are performed three-dimensionally, it is possible to improve the image quality of the bloodstream images and the tissue images that are displayed at the same time. To raise the frame rate, the controlling unit 18 may implement one or both of the parallel simultaneous reception and the increase of the quantity of sections. Also, when the scans are performed two-dimensionally as explained in the first embodiment, the controlling unit 18 may implement one or both of the parallel simultaneous reception and the increase of the quantity of sections, for the purpose of raising the frame rate.

In the first to the third embodiments, the examples are explained in which the first ultrasound scan implementing the high frame rate method is performed for the purpose of obtaining the bloodstream information. However, the first ultrasound scan implementing the high frame rate method is also applicable to the TDI or the elastography described above. More specifically, any reflected-wave signals from a moving object with motion are usable as the Doppler information. Accordingly, the processes described in the first to the third embodiments are applicable, even if the information related to the motion of a moving object is information related to motion of a tissue. In other words, the controlling unit 18 may cause an ultrasound scan to acquire Doppler image data of a tissue to be performed as the first ultrasound scan. Alternatively, the controlling unit 18 may cause an ultrasound scan to acquire elastography to be performed as the first ultrasound scan.

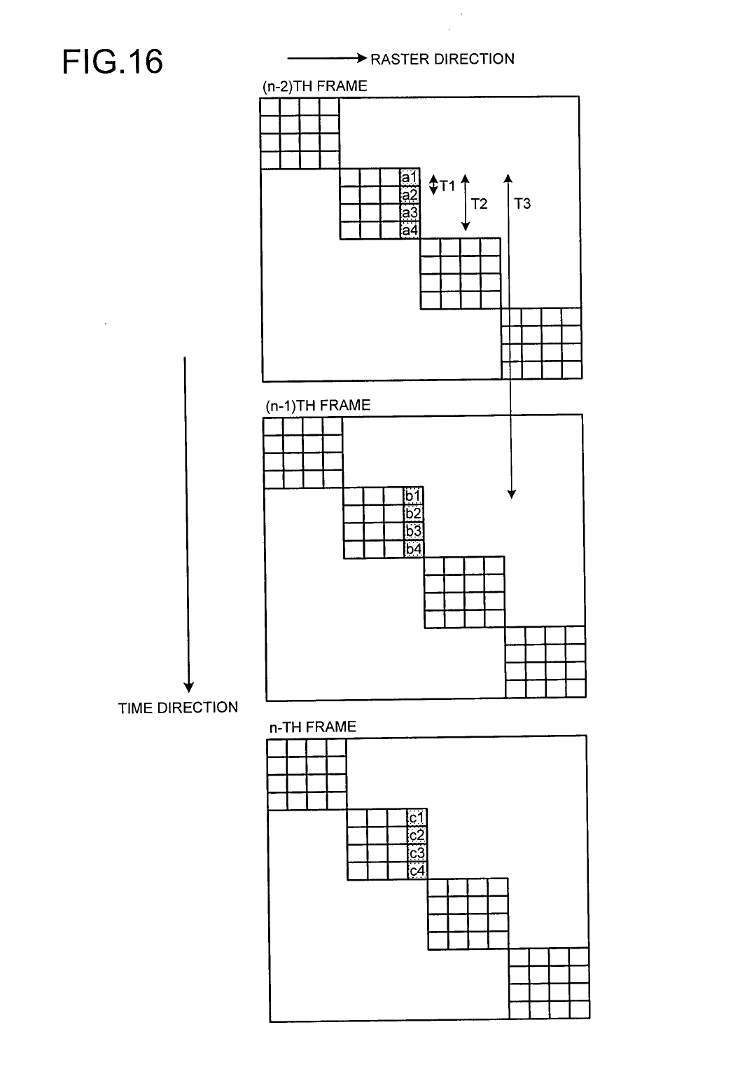

FIGS. 14A and 14B are drawings for explaining a fourth embodiment. In the fourth embodiment, when the ultrasound diagnosis apparatus is set in a tissue Doppler mode, the monitor 2 displays, under control of the controlling unit 18, the B-mode images (the tissue images) on the left side, while displaying superimposed images on the right side in which the B-mode images and tissue Doppler images are superimposed together, as shown in FIG. 14A.

Further, according to the fourth embodiment, when the ultrasound diagnosis apparatus is set in an elastography mode, the monitor 2 displays, under control of the controlling unit 18, the B-mode images (the tissue images) on the left side, while displaying superimposed images on the right side in which the B-mode images and elastography images are superimposed together, as shown in FIG. 14B.

According to the fourth embodiment, it is possible to improve the image quality of the images indicating the motion information of the tissue and the tissue images that are displayed at the same time.

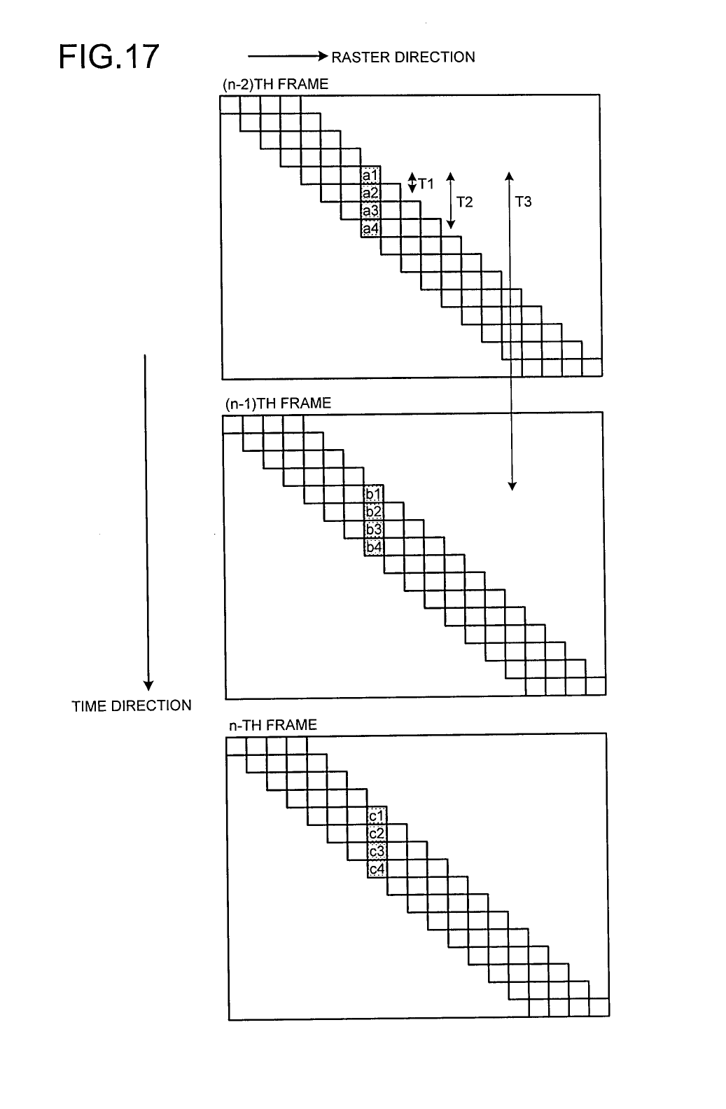

In a fifth embodiment, an example will be explained in which an ultrasound scan that is in a mode different from that of the first ultrasound scan explained in the first to the fourth embodiments is performed as the first ultrasound scan, with reference to FIGS. 15 to 17. FIGS. 15 to 17 are drawings for explaining the fifth embodiment.

In the first ultrasound scan described in the first to the fourth embodiments, the reflected waves are received by performing an ultrasound transmission/reception once for any one scanning line, so as to obtain the reflected-wave data (the reception signals) generated from the received reflected waves. Thus, the reception signals are obtained from the scanning lines structuring the first scanned region. Further, the Doppler processing unit 14 generates the Doppler data by performing the MTI filtering process (e.g., the IIR filtering process) on the data sequence including the reception signals in the latest frame and the groups of reception signals corresponding to a certain number of past frames, for each of the scanning lines.

Similarly to the first ultrasound scan described in the first to the fourth embodiments, the first ultrasound scan according to a fifth embodiment is an ultrasound scan according to a method for performing a high pass filtering process on the data sequence along the frame direction. However, the controlling unit 18 according to the fifth embodiment causes an ultrasound scan in which an ultrasound transmission/reception is performed multiple times for each of the scanning lines, to be performed as the first ultrasound scan. Further, under control of the controlling unit 18 according to the fifth embodiment, the transmitting and receiving unit 11 or the Doppler processing unit 14 performs a signal averaging process on the plurality of reception signals from each of the scanning lines. As a result, a reception signal for each of the plurality of scanning lines structuring the first scanned range is obtained. After that, the Doppler processing unit 14 generates the Doppler data by performing a high pass filtering process on the data sequence along the frame direction.

In the first ultrasound scan according to the fifth embodiment, first, a plurality of reception signals are obtained from any one scanning line. After that, in the first ultrasound scan according to the fifth embodiment, a signal averaging process is performed on the plurality of reception signals obtained from any one scanning line so that one reception signal is eventually output for each scanning line. The plurality of reception signals on which the signal averaging process is performed are signals having phase information, such as the IQ signals and the RF signals. In other words, the signal averaging process performed in the fifth embodiment is a coherent addition process. By performing the coherent addition process, it is possible to improve the signal/noise (S/N) ratio of the reception signals. As a result, according to the fifth embodiment, for example, it is possible to improve the S/N ratio of the color Doppler image data.

For example, in the first ultrasound scan according to the fifth embodiment, an ultrasound transmission/reception is performed four times for each of the scanning lines structuring the first scanned region. After that, in the first ultrasound scan according to the fifth embodiment, for example, the signal averaging process is performed on the four sets of pieces of reflected-wave data (the reception signals) obtained from any one scanning line, so that one reception signal is eventually output for each scanning line. For example, by performing the signal averaging process on the four sets of reception signals, the S/N ratio is improved by “6 dB”.

It should be noted, however, that in the first ultrasound scan described above, because the ultrasound transmission/reception is performed four times for each of the scanning lines during the ultrasound scan corresponding to one frame, the frame rate becomes low. To cope with this situation, it is also acceptable to configure the first ultrasound scan according to the fifth embodiment, so that the controlling unit 18 causes a parallel simultaneous reception to be performed when the ultrasound transmission/reception is performed multiple times for each of the scanning lines structuring the first scanned region. An example of the first ultrasound scan to which the parallel simultaneous reception explained in the third embodiment is applied will be explained with reference to FIG. 15, before explaining an example in which the first ultrasound scan according to the fifth embodiment is performed with a parallel simultaneous reception.

In FIG. 15, the raster direction (the scanning direction) extends in the left-and-right direction, whereas the time direction (the frame direction) extends in the up-and-down direction. In the example shown in FIG. 15, the quantity of scanning lines (i.e., the raster number) structuring the first scanned region is “16”, while reflected waves in four directions are simultaneously received by performing a parallel simultaneous reception. Further, in the example shown in FIG. 15, because the quantity of scanning lines is “16”, whereas the parallel simultaneous reception number is “4”, the first scanned region is divided into four regions (a first region, a second region, a third region, and a fourth region) each structured with four scanning lines.

The ultrasound probe 1 performs an ultrasound transmission by using the center of the first region in the raster direction as a transmission scanning line and simultaneously receives reflected waves from the scanning lines in the four directions structuring the first region. As a result, reception signals from the four scanning lines in the first region are generated. The same process is performed for the second region, the third region, and the fourth region. As a result, reception signals from the sixteen scanning lines structuring the first scanned region are obtained. In FIG. 15, “A”, “B”, and “C” indicate the reception signals from mutually the same scanning line in “the (n−2)th frame, the (n−1)th frame, and the n-th frame”, respectively. The Doppler processing unit 14 performs the MTI filtering process on the data sequence “A, B, and C” in mutually the same location in these consecutive frames.

In contrast, when a parallel simultaneous reception is applied to the first ultrasound scan according to the fifth embodiment, the controlling unit 18 implements either a first method or a second method. According to the first method, the controlling unit 18 causes a parallel simultaneous reception to be performed by dividing the first scanned region into a plurality of regions in such a manner that none of regions that are positioned adjacent to each other overlaps each other. According to the second method, the controlling unit 18 causes a parallel simultaneous reception to be performed by dividing the first scanned region into a plurality of regions in such a manner that any regions that are positioned adjacent to each other overlap each other.

FIG. 16 is a drawing of an example in which the parallel simultaneous reception is applied to the first ultrasound scan according to the fifth embodiment, on the basis of the first method. FIG. 17 is a drawing of an example in which the parallel simultaneous reception is applied to the first ultrasound scan according to the fifth embodiment, on the basis of the second method.

In FIGS. 16 and 17, similarly to the example explained with reference to FIG. 15, the raster direction (the scanning direction) extends in the left-and-right direction, whereas the time direction (the frame direction) extends in the up-and-down direction. Further, in FIGS. 16 and 17, similarly to the example explained with reference to FIG. 15, the quantity of scanning lines (i.e., the raster number) structuring the first scanned region is “16”, while reflected waves in four directions are simultaneously received by performing the parallel simultaneous reception. In FIGS. 16 and 17, “T1” denotes the sampling time period. In FIGS. 16 and 17, “T2” denotes the added width. In FIGS. 16 and 17, “T3” denotes the frame time period. The frame time period “T3” corresponds to a pulse repetition time period in an ordinary Doppler mode.

According to the first method, as illustrated in FIG. 16, similarly to the example shown in FIG. 15, the first scanned region is divided into four regions (a first region, a second region, a third region, and a fourth region) each structured with four scanning lines. According to the first method, however, it should be noted that the parallel simultaneous reception is repeated four times for each of the regions, as illustrated in FIG. 16. As a result, as illustrated in FIG. 16, four sets of reception signals in mutually the same location from mutually the same reception scanning line are obtained in the (n−2)th frame. In FIG. 16, these four sets of pieces of data are marked as “a1, a2, a3, and a4”. Similarly, as illustrated in FIG. 16, four sets of reception signals in mutually the same location from mutually the same reception scanning line are obtained in the (n−1)th frame. In FIG. 16, these four sets of pieces of data are marked as “b1, b2, b3, and b4”. Similarly, as illustrated in FIG. 16, four sets of reception signals in mutually the same location from mutually the same reception scanning line are obtained in the n-th frame. In FIG. 16, these four sets of pieces of data are marked as “c1, c2, c3, and c4”.

For example, the transmitting and receiving unit 11 outputs “A=(a1+a2+a3+a4)/4”. Further, for example, the transmitting and receiving unit 11 outputs “B=(b1+b2+b3+b4)/4”. Further, for example, the transmitting and receiving unit 11 outputs “C=(c1+c2+c3+c4)/4”. As a result, the S/N ratio is improved by “6 dB” in comparison to the ratio prior to the signal averaging process. Further, the Doppler processing unit 14 performs the MTI filtering process on the data sequence “A, B, and C” in mutually the same location in the consecutive frames.

In terms of the Doppler frequency, a low pass filter is applied as a result of adding together the four pieces of data. However, because the velocity component that is cut off due to the sampling time period “T1” and the added width “T2” has a sufficiently higher velocity compared to the frame time period “T3”, no problem occurs in viewing moving objects having a low flow rate.

According to the second method, as shown in FIG. 17 for example, a four-direction parallel simultaneous reception is performed by staggering the position of the transmission scanning line by one scanning line at a time. As a result, similarly to the first method, as shown in FIG. 17, four sets of reception signals “a1, a2, a3, and a4” in mutually the same location from mutually the same reception scanning line are obtained in the (n−2)th frame, so that “A=(a1+a2+a3+a4)/4” is output. Further, similarly to the first method, as shown in FIG. 17, four sets of reception signals “b1, b2, b3, and b4” in mutually the same location from mutually the same reception scanning line are obtained in the (n−1)th frame, so that “B=(b1+b2+b3+b4)/4” is output. Further, similarly to the first method, as shown in FIG. 17, four sets of reception signals “c1, c2, c3, and c4” in mutually the same location from mutually the same reception scanning line are obtained in the n-th frame, so that “C=(c1+c2+c3+c4)/4” is output. As a result, the S/N ratio is improved by “6 dB” in comparison to the ratio prior to the signal averaging process. In FIGS. 16 and 17, the frame rate of the Doppler image data is the same.

In the example shown in FIG. 17, for a scanning line from which only two sets of reception signals are obtained, the signal averaging process is performed on the two sets of reception signals. For a scanning line from which only three sets of reception signals are obtained, the signal averaging process is performed on the three sets of reception signals. Further, in the example shown in FIG. 17, for a scanning line from which only one set of reception signals is obtained, these reception signals is the data that serves as a processing target of the Doppler processing unit 14. Further, according to the second method, for example, the position of the transmission scanning line may be staggered by two scanning lines at a time, in accordance with the number of sets of reception signals used as the target of the signal averaging process.

An advantage of implementing the second method will be explained below. When the first method is implemented, in the first ultrasound scan, the regions on which the parallel simultaneous reception is performed multiple times do not overlap each other. According to the first method illustrated in FIG. 16, because the transmission positions for obtaining the four reception signals from mutually the same scanning line is the same, the phase of the transmission beams does not change. However, in the first method illustrated in FIG. 16, the regions on which the parallel simultaneous reception is performed four times do not overlap each other. For this reason, according to the first method illustrated in FIG. 16, a striped artifact may occur between the regions each corresponding to four raster units.

In contrast, when the second method is implemented, in the first ultrasound scan, the parallel simultaneous reception is performed once for each of the regions that are arranged so that the regions positioned adjacent to each other overlap each other. According to the second method illustrated in FIG. 17, because the transmission positions for obtaining the four reception signals from mutually the same scanning line varies, a minor phase shift occurs. However, it is possible to eliminate such a phase shift by using an MTI filter. Further, according to the second method illustrated in FIG. 17, because the regions on which the parallel simultaneous reception is performed are arranged so to overlap each other by three scanning lines, no striped artifact occurs.

As explained above, according to the fifth embodiment, the HPF process in the frame direction is performed by using the reception signals resulting from the coherent addition process performed on the plurality of reception signals obtained from each of the scanning lines. As a result, according to the fifth embodiment, although the frame rate becomes lower than those in the first ultrasound scan explained in the first to the fourth embodiments, it is possible to improve the S/N ratio of the reception signals used for generating the images indicating the moving object information. In the description above, the example in which the parallel simultaneous reception number is “4” is explained; however, the parallel simultaneous reception number may be set to an arbitrary value. Further, as explained at first, the first ultrasound scan according to the fifth embodiment is also possible even if no parallel simultaneous reception is performed. Further, another arrangement is also acceptable in which, under the control of the controlling unit 18 according to the fifth embodiment, the transmitting and receiving unit 11 or the Doppler processing unit 14 performs an LPF process similar to the signal averaging process on the plurality of reception signals obtained from each of the scanning lines. Further, the configurations explained in the first to the fourth embodiments are also applicable to the fifth embodiment, except that the mode of the first ultrasound scan is different.

The constituent elements of the apparatuses that are shown in the drawings in relation to the description of the exemplary embodiments are based on functional concepts. Thus, it is not necessary to physically configure the elements as indicated in the drawings. In other words, the specific mode of distribution and integration of the apparatuses is not limited to the ones shown in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a Central Processing Unit (CPU) and a computer program that is analyzed and executed by the CPU or may be realized as hardware using wired logic.

Further, the controlling methods related to the ultrasound scans described in the first to the fifth embodiments may be realized by causing a computer such as a personal computer or a workstation to execute a control computer program (hereinafter, the “control program”) prepared in advance. The control program may be distributed via a network such as the Internet. Furthermore, it is also possible to record the control program onto a computer-readable non-transitory recording medium such as a hard disk, a flexible disk (FD), a Compact Disk Read-Only Memory (CD-ROM), a Magneto-optical (MO) disk, a Digital Versatile Disk (DVD), or a flash memory such as a Universal Serial Bus (USB) memory or a Secure Digital (SD) card memory, so that a computer is able to read the control program from the non-transitory recording medium and to execute the read control program.

As explained above, according to at least one aspect of the first to the fifth embodiments, it is possible to improve the image quality of the images indicating the moving object information and the tissue images that are displayed at the same time.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
an ultrasound probe configured to transmit and receive an ultrasound wave; and
a controlling unit configured to cause the ultrasound probe to perform a first ultrasound scan to obtain information related to motion of a moving object within a first scanned region and causes the ultrasound probe to perform, as a second ultrasound scan to obtain information about a tissue form within a second scanned region, an ultrasound scan in each of a plurality of sectioned regions into which the second scanned region is divided, in a time-division manner between the first ultrasound scans, wherein
as the first ultrasound scan, the controlling unit causes the ultrasound scan to be performed according to a method for obtaining the information related to the motion of the moving object by which a high pass filtering process is performed along a frame direction on reception signals obtained from a plurality of scanning lines structuring the first scanned region.

2. The ultrasound diagnosis apparatus according to claim 1, wherein, as the first ultrasound scan, the controlling unit causes the ultrasound scan to be performed according to a method for obtaining a data sequence along the frame direction by which the reception signals are obtained from the plurality of scanning lines structuring the first scanned region by performing an ultrasound transmission/reception once for each of the scanning lines, so that the high pass filtering process is performed on the obtained reception signals.

3. The ultrasound diagnosis apparatus according to claim 1, wherein, as the first ultrasound scan, the controlling unit causes the ultrasound scan to be performed according to a method for obtaining a data sequence along the frame direction by which the reception signals are obtained from the plurality of scanning lines structuring the first scanned region, either by performing a signal averaging process or by performing a low pass filtering process similar to the signal averaging process on the plurality of reception signals obtained from each of the scanning lines by performing an ultrasound transmission/reception multiple times for each of the scanning lines, so that the high pass filtering process is performed on the obtained reception signals.

4. The ultrasound diagnosis apparatus according to claim 3, wherein, during the first ultrasound scan, the controlling unit causes a parallel simultaneous reception to be performed when an ultrasound transmission/reception is performed multiple times for each of the scanning lines structuring the first scanned region.

5. The ultrasound diagnosis apparatus according to claim 4, wherein the controlling unit either causes the parallel simultaneous reception to be performed by dividing the first scanned region into a plurality of regions or causes the parallel simultaneous reception to be performed by dividing the first scanned region into a plurality of regions in such a manner that regions positioned adjacent to each other overlap each other.

6. The ultrasound diagnosis apparatus according to claim 1, wherein the controlling unit arranges the first ultrasound scans to be performed at regular intervals, by arranging time periods required by sectioned scans of the second ultrasound scan to be equal to one another.

7. The ultrasound diagnosis apparatus according to claim 1, wherein the controlling unit exercises control so that a plurality of pieces of image data in the first scanned region that have been generated by the first ultrasound scan are output as one piece of image data, in accordance with a time period required by the first ultrasound scan performed at one time and a display frame rate.

8. The ultrasound diagnosis apparatus according to claim 1, wherein the controlling unit causes a parallel simultaneous reception to be performed in one or both of the first ultrasound scan and the second ultrasound scan.

9. The ultrasound diagnosis apparatus according to claim 1, wherein, the controlling unit causes an ultrasound scan to acquire either Doppler image data or elastography to be performed as the first ultrasound scan.

10. A controlling method comprising:
a step performed by a controlling unit to cause an ultrasound probe configured to transmit and receive an ultrasound wave to perform a first ultrasound scan to obtain information related to motion of a moving object within a first scanned region and to cause the ultrasound probe to perform, as a second ultrasound scan to obtain information about a tissue form within a second scanned region, an ultrasound scan in each of a plurality of sectioned regions into which the second scanned region is divided, in a time-division manner between the first ultrasound scans, wherein
as the first ultrasound scan, the controlling unit causes the ultrasound scan to be performed according to a method for obtaining the information related to the motion of the moving object by which a high pass filtering process is performed along a frame direction on reception signals obtained from a plurality of scanning lines structuring the first scanned region.
